(19) United States
(12) Patent Application Publication
(10) Pub. No.: US 2014/0088034 A1
(43) Pub. Date: Mar. 27, 2014

(54) NITRIC OXIDE/cGMP PATHWAY INHIBITION OF VLA-4 RELATED CELL ADHESION

(71) Applicant: STC.UNM, Albuquerque, NM (US)

(72) Inventors: Alexandre Chigaev, Santa Fe, NM (US); Larry A. Sklar, Albuquerque, NM (US)

(21) Appl. No.: 14/115,170

(22) PCT Filed: May 10, 2012
(86) PCT No.: PCT/US12/37352

§ 371 Date: Nov. 1, 2013

Related U.S. Application Data
(60) Provisional application No. 61/484,927, filed on May 11, 2011.

(51) Int. Cl. A61K 31/708; A61K 31/132

(52) U.S. Cl. CPC A61K 31/708; A61K 31/132
USPC 514/48; 514/611

(57) ABSTRACT

The invention provides methods of treating nitric oxide/cGMP pathway-cell adhesion disorders and related pharmaceutical compositions, diagnostics, screening techniques and kits. In one embodiment, the invention relates to a method for down-regulating α₄β₁-integrin affinity and inhibiting and reversing adhesion formation in patients or subjects in need using a nitric oxide donor.

### RELATED APPLICATIONS

This application claims priority from U.S. Provisional Application Ser. No. 61/484,927, filed May 11, 2011, entitled “Nitric oxide/cGMP pathway signaling actively down-regulates alpha4beta1-integrin affinity; an unexpected mechanism for inducing cell de-adhesion”, the complete disclosure of which is hereby incorporated by reference in its entirety.

### GOVERNMENT SUPPORT

The invention described herein was funded in part by National Institutes of Health Grant HL081062. Accordingly, the United States has certain rights in the invention.

### FIELD OF THE INVENTION

The invention provides methods of treating nitric oxide/cGMP pathway-cell adhesion disorders and related pharmaceutical compositions, diagnostics, screening techniques and kits. In one embodiment, the invention relates to a method for down-regulating α₄β₁-integrin affinity and inhibiting and reversing adhesion formation in patients or subjects in need using a nitric oxide donor.

### BACKGROUND OF THE INVENTION

Integrin activation in response to inside-out signaling serves as the basis for rapid leukocyte arrest on endothelium, migration, and mobilization of immune cells. Integrin-dependent adhesion is controlled by the conformational state of the molecule, which is regulated by seven-transmembrane Guanine nucleotide binding Protein-Coupled Receptors (GPCRs). α₄β₁-integrin (CD49d/CD29, Very Late Antigen-4, VLA-4) is expressed on leukocytes, hematopoietic progenitors, stem cells, hematopoietic cancer cells, and others. VLA-4 conformation is rapidly up-regulated by inside-out signaling through Gαᵢ-coupled GPCRs and down-regulated by Gαₛ-coupled GPCRs.

Thus, integrins are ubiquitous cell adhesion molecules that play an essential role in the regulation of leukocyte traffic, stem cell mobilization and homing, immune responses, development, hemostasis, and cancer [1-3]. On the cell surface at rest, a variety of integrin exhibit a non-adhesive inactive state and multiple signaling cascades are capable of rapidly and reversibly regulating integrin-dependent cell adhesion. Typically, this regulation is achieved without altering the integrin expression level. Conformational changes within the molecule, together with a spatial reorganization of integrins, are responsible for the rapid modulation of cell adhesion [1, 4-6]. Understanding signaling pathways that regulate activation and, especially, inactivation of integrin-mediated cell adhesion is crucial, as integrins are implicated in many human diseases [7-9]. Several existing and emerging drugs for treating inflammatory diseases, anti-angiogenic cancer therapy, anti-thrombotic therapy, and others specifically target integrin molecules [10-12]. Moreover, interfering with integrin activation by targeting “the final steps of activation process” is envisioned as a novel approach for therapeutic intervention in integrin-related pathologies [13].

Very Late Antigen-4, VLA-4, (α₄β₁-integrin, CD49d/CD29) is expressed on a majority of peripheral blood leukocytes, hematopoietic progenitors and stem cells, as well as hematopoietic cancer cells [2, 14, 15]. VLA-4 has the potential to exhibit multiple affinity (conformational) states that mediate tethering, rolling, and firm arrest on VCAM-1 (CD106, Vascular Cell Adhesion Molecule-1) [16-18]. The VLA-4 conformational state is regulated by G protein-coupled receptors (GPCRs) that operate as receptors for multiple chemokines and chemoattractants. The majority of receptors activating VLA-4 are Gαᵢ-coupled GPCRs that function by inhibiting adenylate cyclase and inducing calcium mobilization. These include CXCR2, CXCR4, and others [19]. Gαᵢ-coupled GPCRs activate integrin by triggering the so-called inside-out signaling pathway [20], which leads to a rapid increase in ligand binding affinity that is translated into the “rapid development of firm adhesion” [18].

Recently, in addition to the inside-out integrin activation pathway, we described a de-activation signaling pathway that can rapidly down-regulate the binding affinity state of the VLA-4 binding pocket. Two Gαₛ-coupled GPCRs (histamine H2 receptor and β2-adrenergic receptors), an adenylyl cyclase activator, and a cell permeable analog of cAMP showed the ability to regulate VLA-4 ligand binding affinity as well as VLA-4/VCAM-1 dependent cell adhesion on live cells in real-time [21].

Both cAMP/PKA and cGMP/PKG signaling pathways play an inhibitory role in GPCR-induced platelet aggregation and adhesion [22], which is known to be critically dependent on the activation state of platelet integrins [23, 24]. Cyclic nucleotide dependent kinases (PKA and PKG) share a strong sequence homology and exhibit overlapping substrate specificity [25]. Nitric oxide signaling is critical for hematopoietic progenitor and stem cell mobilization [26, 27], a physiological process that is critically dependent on the interaction between VLA-4 integrin and VCAM-1 [28-32]. Nitric oxide is also shown to antagonize GPCR signaling in muscle cells [33]. The molecular mechanism by which nitric oxide regulates integrin-dependent adhesion is under active investigation. Several reports indicate that direct s-nitrosylation of cytoskeletal proteins [34], or integrins themselves [35], can be involved in the regulation of integrin-dependent adhesion.

An understanding of the effects of exogenous nitric oxide and other cGMP pathway regulators on VLA-4 conformational regulation would yield improved methods of treating and diagnosing wide variety of disorders implicated by VLA-4-related cell adhesion.

### SUMMARY OF THE INVENTION

We have discovered that nitric oxide/cGMP signaling pathway can actively down-regulate VLA-4 affinity, even under conditions of constant signaling. The nitric oxide/cGMP signaling pathway can rapidly down-modulate the affinity state of the VLA-4 binding pocket, especially under the condition of sustained Gαᵢ-coupled GPCR signalling generated by a non-desensitizing receptor mutant. This suggests a fundamental role of this pathway in de-activation of integrin-dependent cell adhesion. Our finding that NO/cGMP pathway directly regulates integrin-dependent immune cell adhesion enables the repositioning of existing drugs toward pathologies where integrin-mediated excessive immune cell adhesion/recruitment is envisioned to be detrimental.

Accordingly, in one embodiment, the invention provides a method of treating a subject who suffers from or is at risk of developing a VLA-4-related cell adhesion disorder as defined hereinafter, the method comprising administering to the subject a pharmaceutically-effective amount of a nitric oxide/cGMP signaling pathway modulator selected from the group consisting of a nitric oxide donor, a nitric oxide-independent activator of soluble guanylyl cyclase, or a cell permeable analog of cGMP.

In one embodiment of this method:

(a) the nitric oxide (NO) donor is (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazenium diolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF;
(b) the nitric oxide-independent activator of soluble guanylyl cyclase is selected from the group consisting of BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin; and
(c) the cell permeable analog of cGMP is N2,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

In certain embodiments, the subject suffering from or at risk of developing a VLA-4-related cell adhesion disorder is co-administered a combination of at least two active ingredients selected from the group consisting of a nitric oxide donor, a nitric oxide-independent activator of soluble guanylyl cyclase, and a cell permeable analog of cGMP.

In another embodiment, the VLA-4-related cell adhesion disorder is a cancer as described hereinafter and the subject is co-administered: (1) at least one active ingredient selected from the group consisting of a nitric oxide donor, a nitric oxide-independent activator of soluble guanylyl cyclase, and a cell permeable analog of cGMP; and (2) at least one additional anti-cancer agent.

In still another embodiment, the invention provides a method of treating a subject who has been diagnosed as suffering from at least one VLA-4-related cell adhesion disorder selected from the group consisting of multiple sclerosis, ulcerative colitis, Crohn's disease, rheumatoid arthritis, asthma, acute juvenile onset diabetes (Type 1), AIDS dementia, atopic dermatitis, psoriasis, nephritis, retinitis, acute leukocyte-mediated lung injury, transplant rejection, and graft versus host disease the method comprising treating the at least one VLA-4-related cell adhesion disorder by administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP.

In still another embodiment, the invention provides a method of treating a subject who has been diagnosed as suffering from at least one VLA-4-related cell adhesion disorder selected from the group consisting of atherosclerosis and myocardial ischemia, the method comprising treating the at least one VLA-4-related cell adhesion disorder by administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP. The subject diagnosed with atherosclerosis and myocardial ischemia may also suffer from an additional cardiac disorder selected from the group consisting of decompensated heart failure, arterial pulmonary hypertension, venous pulmonary hypertension, hypoxic pulmonary hypertension, thromboembolic pulmonary hypertension and miscellaneous pulmonary hypertension, and the additional cardiac disorder may be treated by separately administering one of the nitric oxide/cGMP signaling pathway modulators.

In still another embodiment, the invention provides a method of treating a subject who has been diagnosed as suffering from at least one VLA-4-related cell adhesion disorder selected from the group consisting of tumor metastasis, melanoma, multiple myeloma, malignant lymphoma, acute and chronic leukemias, pancreatic cancer, neuroblastoma, small cell and non-small cell lung cancer, mesothelioma, colorectal carcinoma, and breast cancer, the method comprising treating the at least one VLA-4-related cell adhesion disorder by administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP. For example, the diagnosed tumour metastasis, melanoma, multiple myeloma, malignant lymphoma, acute and chronic leukemias, pancreatic cancer, neuroblastoma, small cell and non-small cell lung cancer, mesothelioma, colorectal carcinoma, or breast cancer is treated by administering to the subject one or more nitric oxide/cGMP signaling pathway modulators selected from the group consisting of BAY 41-2272, BAY 41-8543, BAY 58-2667 (cinaciguat), BAY 60-2770, BAY 63-2521, YC-1 (3-(5′-hydroxymethyl-2′-furyl)-1-benzylindazole), A-350619, A-344905, and A-778935. An additional anti-cancer agent can be co-administered to the subject.

In still another embodiment, the invention provides a method of treating a subject who has been diagnosed as suffering from a non-metastatic cancer, the method comprising administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP to prevent metastasis of the cancer. For example, to prevent metastasis, the subject who has been diagnosed as suffering from a non-metastatic cancer may be treated with one or more nitric oxide/cGMP signaling pathway modulators selected from the group consisting of BAY 41-2272, BAY 41-8543, BAY 58-2667 (cinaciguat), BAY 60-2770, BAY 63-2521, YC-1 (3-(5′-hydroxymethyl-2′-furyl)-1-benzylindazole), A-350619, A-344905, and A-778935.

In still another embodiment, the invention provides a method of determining whether a subject suffers from, or is at risk of developing VLA-4-related cell adhesion disorder, the method comprising determining a cyclic GMP (cGMP) level in a sample obtained from the subject and comparing the determined cyclic GMP (cGMP) level to a control cyclic GMP (cGMP) level, wherein a decrease in cyclic GMP (cGMP) level indicates an increased likelihood that the subject suffers from or is at risk of developing VLA-4-related cell adhesion disorder. For example, this method can comprise the steps of:

(a) contacting a biological test sample obtained from the subject with an antibody or an antigen binding fragment thereof having specific binding affinity for cGMP, under conditions such that a complex can form between cGMP and the antibody or the antigen binding fragment thereof;
(b) measuring the amount of said complex, thereby determining the amount of cGMP in said biological test sample; and
(c) comparing the amount of cGMP in said biological test sample to a standard or control sample;
wherein a decreased amount of cGMP in said biological test sample relative to the standard or control sample is indicative of VLA-4-related cell adhesion disorder in said test sample.

In the method described above, the amount of cGMP can be determined by a variety of techniques, including immunohistochemistry, immunostaining, immunofluorescence and western blot assay. Also, the method can use monoclonal or polyclonal antibodies.

In still another embodiment, the invention provides a method of screening for a composition useful in the treatment of a VLA-4-related cell adhesion disorder, the method comprising contacting a sample of a cell population evidencing a VLA-4-related cell adhesion morphology with a candidate composition and determining the extent to which the candidate composition up-regulates translation of cyclic GMP (cGMP), wherein the candidate composition is identified as being potentially useful in the treatment of a VLA-4-related cell adhesion disorder if translation levels of cyclic GMP (cGMP) in the sample are greater than the comparable control values for an untreated cell population evidencing a VLA-4-related cell adhesion morphology (e.g. VLA-4 dependent cell aggregation). For example, this method can comprise the steps of:

(a) contacting a first sample of a VLA-4-related cell adhesion disorder cell population with a candidate composition;
(b) determining one or more values representing the extent to which the candidate composition up-regulates translation of cGMP in the first sample; and
(c) comparing the determined one or more values to control values based on translation levels of cGMP in a second, untreated sample of the cell population, wherein the candidate composition is identified as being potentially useful in the treatment of a VLA-4-related cell adhesion disorder if translation levels of cGMP in the first sample are greater than the comparable control values in the second sample.

In still another embodiment, the invention provides a kit comprising:

(a) at least one reagent which is selected from the group consisting of (i) reagents that detect a transcription product of the gene coding for a cGMP protein marker (ii) reagents that detect a translation product of the gene coding for cGMP, and/or reagents that detect a fragment or derivative or variant of said transcription or translation product;
(b) instructions for diagnosing, or prognosticating a VLA-4-related cell adhesion disorder, or determining the propensity or predisposition of a subject to develop a VLA-4-related cell adhesion disorder or of monitoring the effect of a treatment of a VLA-4-related cell adhesion disorder.

In still another embodiment, the invention provides a pharmaceutical composition comprising:

(a) at least one nitric oxide/cGMP signaling pathway modulator as defined herein;
(b) at least one additional VLA-4 antagonist as defined herein; and optionally
(b) a pharmaceutically-acceptable excipient.

In still another embodiment, the invention provides a pharmaceutical composition comprising:

(a) at least one nitric oxide/cGMP signaling pathway modulator as defined herein;
(b) at least one additional anti-cancer agent as defined herein; and optionally
(b) a pharmaceutically-acceptable excipient.

In still another embodiment, the invention provides a method of regulation of stem cell adhesion that includes (but not limited to) cell mobilization into the peripheral blood, for example, for the purpose of autologous or heterologous stem cell transplantation, or other therapies that require stem cell collection. The method comprises administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP. Because VLA-4 integrin is specifically responsible for the retention and homing of stem/progenitor cells into the peripheral blood, VLA-4 affinity down modulation leads to stem cell mobilization. Thereafter, cells optionally can be collected, purified, as needed, and/or transplanted.

By elucidating the roles of exogenous nitric oxide and other cGMP pathway regulators on VLA-4 conformational regulation, we have discovered improved methods of treating and diagnosing wide variety of disorders implicated by VLA-4-related cell adhesion. These and other aspects are described further in the Detailed Description of the Invention.

### BRIEF DESCRIPTION OF THE FIGURES

FIG. 1 illustrates that, as determined in the experiments of Example 1, the following two molecules stimulate the three initial consecutive steps of the nitric oxide/cGMP signaling pathway in leukocytes and therefore can be used to mimic nitric oxide/cGMP signaling in leukocytes: (1) BAY 41-2272, which is an activator of soluble guanylyl cyclase, which stimulates cGMP production through an NO-independent mechanism [39, 40]; and (2) N²,2′-O-dibutyrylguanosine 3′,5′-cyclic monophosphate, which is a cell permeable cGMP analog that activates protein kinase G [41].

FIG. 2 illustrates the effect of nitric oxide addition on binding and dissociation of the LDV-FITC probe on U937 cells, treated with different Gαᵢ-coupled receptor ligands, as determined in accordance with the experiments of Example 2.

FIG. 3 illustrates the effect of guanylyl cyclase activator on binding and dissociation of the LDV-FITC probe on U937 cells, treated with different Gαᵢ-coupled receptor ligands, as determined in accordance with the experiments of Example 3.

FIG. 4 illustrates the effect of the cell permeable analog of cGMP on binding and dissociation of the LDV-FITC probe on U937 cells stably transfected with the non-desensitizing mutant of FPR, as determined in accordance with the experiments of Example 4.

FIG. 5 illustrates changes in cell adhesion between U937 FPR (ΔST) and VCAM-1-transfected B78H1 cells in the resting state and in response to receptor stimulation, as determined in accordance with the experiments of Example 4.

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

As used herein, “antibody” includes, but is not limited to, monoclonal antibodies. The following disclosure from U.S. Patent Application Document No. 20100284921, the entire contents of which are hereby incorporated by reference, exemplifies techniques that are useful in making antibodies employed in formulations of the instant invention.

As described in U.S. Patent Application Document No. 20100284921, “antibodies . . . may be polyclonal or monoclonal. Monoclonal antibodies are preferred. The antibody is preferably a chimeric antibody. For human use, the antibody is preferably a humanized chimeric antibody.

An anti-target-structure antibody may be monovalent, divalent or polyvalent in order to achieve target structure binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

The invention also includes [use of] functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application Nos. WO 1993/21319 and WO 1989/09622. Functional equivalents include polypeptides with amino acid sequences substantially the same as the amino acid sequence of the variable or hypervariable regions of the antibodies raised against target integrins according to the practice of the present invention.

Functional equivalents of the anti-target-structure antibodies further include fragments of antibodies that have the same, or substantially the same, binding characteristics to those of the whole antibody. Such fragments may contain one or both Fab fragments or the F(ab′).sub.2 fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The monoclonal antibodies may be advantageously cleaved by proteolytic enzymes to generate fragments retaining the target structure binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)2 fragment.

Single chain antibodies or Fv fragments are polypeptides that consist of the variable region of the heavy chain of the antibody linked to the variable region of the light chain, with or without an interconnecting linker. Thus, the Fv comprises an antibody combining site.

Hybrid antibodies may be employed. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies (e.g. for preparing an antibody or an antigen binding fragment thereof having specific binding affinity for cGMP or VLA-4 are either described in the experiments herein or are otherwise known to those skilled in the art. See, Goding, “Monoclonal Antibodies Principles and Practice”, Academic Press (1983), p. 119-123. Fragments of the monoclonal antibodies containing the antigen binding site, such as Fab and F(ab′)2 fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the practice of the invention is a polyclonal antibody (IgG), the antibody is generated by inoculating a suitable animal with a target structure or a fragment thereof. Antibodies produced in the inoculated animal that specifically bind the target structure are then isolated from fluid obtained from the animal. Anti-target-structure antibodies may be generated in this manner in several non-human mammals such as, but not limited to, goat, sheep, horse, rabbit, and donkey. Methods for generating polyclonal antibodies are well known in the art and are described, for example in Harlow et al. (In: Antibodies, A Laboratory Manual, 1988, Cold Spring Harbor, N.Y.).

When the antibody used in the methods used in the practice of the invention is a monoclonal antibody, the antibody is generated using any well known monoclonal antibody preparation procedures such as those described, for example, in Harlow et al. (supra) and in Tuszynski et al. (Blood 1988, 72:109-115). Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (supra).

The effects of sensitization in the therapeutic use of animal-origin monoclonal antibodies in the treatment of human disease may be diminished by employing a hybrid molecule generated from the same Fab fragment, but a different Fc fragment, than contained in monoclonal antibodies previously administered to the same subject. It is contemplated that such hybrid molecules formed from the anti-target-structure monoclonal antibodies may be used in the present invention. The effects of sensitization are further diminished by preparing animal/human chimeric antibodies, e.g., mouse/human chimeric antibodies, or humanized (i.e. CDR-grafted) antibodies. Such monoclonal antibodies comprise a variable region, i.e., antigen binding region, and a constant region derived from different species. By ‘chimeric’ antibody is meant an antibody that comprises elements partly derived from one species and partly derived form at least one other species, e.g., a mouse/human chimeric antibody.

Chimeric animal-human monoclonal antibodies may be prepared by conventional recombinant DNA and gene transfection techniques well known in the art. The variable region genes of a mouse antibody-producing myeloma cell line of known antigen-binding specificity are joined with human immunoglobulin constant region genes. When such gene constructs are transfected into mouse myeloma cells, the antibodies produced are largely human but contain antigen-binding specificities generated in mice. As demonstrated by Morrison et al., 1984, Proc. Natl. Acad. Sci. USA 81:6851-6855, both chimeric heavy chain V region exon (VH)-human heavy chain C region genes and chimeric mouse light chain V region exon (VK)-human K light chain gene constructs may be expressed when transfected into mouse myeloma cell lines. When both chimeric heavy and light chain genes are transfected into the same myeloma cell, an intact H2L2 chimeric antibody is produced. The methodology for producing such chimeric antibodies by combining genomic clones of V and C region genes is described in the above-mentioned paper of Morrison et al., and by Boulianne et al. (Nature 1984, 312:642-646). Also see Tan et al. (J. Immunol. 1985, 135:3564-3567) for a description of high level expression from a human heavy chain promotor of a human-mouse chimeric K chain after transfection of mouse myeloma cells. As an alternative to combining genomic DNA, cDNA clones of the relevant V and C regions may be combined for production of chimeric antibodies, as described by Whitte et al. (Protein Eng. 1987, 1:499-505) and Liu et al. (Proc. Natl. Acad. Sci. USA 1987, 84:3439-3443). For examples of the preparation of chimeric antibodies, see the following U.S. Pat. Nos. 5,292,867; 5,091,313; 5,204,244; 5,202,238; and 5,169,939. The entire disclosures of these patents, and the publications mentioned in the preceding paragraph, are incorporated herein by reference. Any of these recombinant techniques are available for production of rodent/human chimeric monoclonal antibodies against target structures.

To further reduce the immunogenicity of murine antibodies, “humanized” antibodies have been constructed in which only the minimum necessary parts of the mouse antibody, the complementarity-determining regions (CDRs), are combined with human V region frameworks and human C regions (Jones et al., 1986, Nature 321:522-525; Verhoeyen et al., 1988, Science 239:1534-1536; Hale et al., 1988, Lancet 2:1394-1399; Queen et al., 1989, Proc. Natl. Acad. Sci. USA 86:10029-10033). The entire disclosures of the aforementioned papers are incorporated herein by reference. This technique results in the reduction of the xenogeneic elements in the humanized antibody to a minimum. Rodent antigen binding sites are built directly into human antibodies by transplanting only the antigen binding site, rather than the entire variable domain, from a rodent antibody. This technique is available for production of chimeric rodent/human anti-target structure antibodies of reduced human immunogenicity.”

Further, standard techniques for growing cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, New York; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press; Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Imaging techniques and diagnostic methods described herein, especially flow cytometry as described in greater detail herein, ss can use fluorescence-inducing compounds, e.g. a fluorescent moiety such as a fluorescein dye or a rhodamine dye. In some embodiments, the fluorescent moiety comprises two or more fluorescent dyes that can act cooperatively with one another, for example by fluorescence resonance energy transfer (“FRET”). The fluorescent moiety may be any fluorophore that is capable of producing a detectable fluorescence signal in an assay medium; the fluorescence signal can be “self-quenched” and capable of fluorescing in an aqueous medium. “Quench” refers to a reduction in the fluorescence intensity of a fluorescent group as measured at a specified wavelength, regardless of the mechanism by which the reduction is achieved. As specific examples, the quenching may be due to molecular collision, energy transfer such as FRET, a change in the fluorescence spectrum (color) of the fluorescent group or any other mechanism. The amount of the reduction is not critical and may vary over a broad range. The only requirement is that the reduction be measurable by the detection system being used. Thus, a fluorescence signal is “quenched” if its intensity at a specified wavelength is reduced by any measurable amount.

Examples of fluorophores include xanthenes such as fluoresceins, rhodamines and rhodols, cyanines, phtalocyanines, squairanines, bodipy dyes, pyrene, anthracene, naphthalene, acridine, stilbene, indole or benzindole, oxazole or benzoxazole, thiazole or benzothiazole, carbocyanine, carbostyryl, prophyrin, salicylate, anthranilate, azulene, perylene, pyridine, quinoline, borapolyazaindacene, xanthene, oxazine or benzoxazine, carbazine, phenalenone, coumarin, benzofuran, or benzphenalenone. Examples of rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichlorotetramethylrhodamine (dTAMRA). Examples of fluorescein dyes include, but are not limited to, 4,7-dichlorofluoresceins, 5-carboxyfluorescein (5-FAM) and 6-carboxyfluorescein (6-FAM).

Detection and spatial localization in a biological sample as described herein may be based on, but not restricted to fluorescence in the ultra-violet, visible, infrared spectral regions, or may report via radiofrequencies (MRI/NMR) and well as radioactive detection. In addition, a reporter group containing heavy atoms is employed for detection using electron microscopy (EM or TEM), scanning EM (SEM) or mass spectral or equivalent techniques. In alternative embodiments, the reporter (domains or moieties) comprise functional groups that either turn off or on its reporting function from its native state, but in the presence of a biological sample (for example; pH change, presence of a specific enzyme, metal etc.) changes its state, giving further details to the biological environment in an autophagic vesicle.

Cell samples used in methods of the invention can be stem cells. Stem cells are cells capable of differentiation into other cell types, including those having a particular, specialized function (i.e., terminally differentiated cells, such as erythrocytes, macrophages, etc.), progenitor (i.e., “multipotent”) cells which can give rise to any one of several different terminally differentiated cell types, and cells that are capable of giving rise to various progenitor cells. Cells that give rise to some or many, but not all, of the cell types of an organism are often termed “pluripotent” stem cells, which are able to differentiate into any cell type in the body of a mature organism, although without reprogramming they are unable to de-differentiate into the cells from which they were derived. “Multipotent” stem/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells (e.g., fertilized oocytes, cells of embryos at the two and four cell stages of development), which have the ability to differentiate into any type of cell of the particular species. For example, a single totipotent stem cell could give rise to a complete animal, as well as to any of the myriad of cell types found in the particular species (e.g., humans). In this specification, pluripotent and totipotent cells, as well as cells with the potential for differentiation into a complete organ or tissue, are referred as “primordial” stem cells.

In addition to the methodologies described herein, “the morphology of positive control cell samples” can be determined using techniques that are well-known to those or ordinary skill in the art. For example, see Danussi, et al., “EMILIN1-α4/α9 integrin interaction inhibits dermal fibroblast and keratinocyte proliferation”, JCB vol. 195 no. 1 131-14 (2011); Conant, et al., “Well plate-coupled microfluidic devices designed for facile image-based cell adhesion and transmigration assays”, 2011; 6(8):e23758. Epub 2011 Aug. 18; J. Biomol. Screen., 2010 January; 15(1):102-6. Epub 2009 Dec. 4; and Sharif, et al., Thrombin-activatable carboxypeptidase B cleavage of osteopontin regulates neutrophil survival and synoviocyte binding in rheumatoid arthritis”, Arthritis Rheum. 2009 October; 60(10):2902-12.

As disclosed herein, the invention enables the use of high-throughput format, high-content imaging to examine the cell sample for a nitric oxide/cGMP signaling pathway modulator effect on a cGMP and/or VLA-4-related cell morphology.

In one embodiment, determination of a nitric oxide/cGMP signaling pathway modulator effect on a cGMP and/or VLA-4-related cell morphology involves detecting the amount of cGMP and/or VLA-4, or the amount of cGMP and/or VLA-4 activity (e.g. cell adhesion), in a sample (e.g. a cell) both in the absence and presence of a candidate composition and an increase or a decrease in the amount of cGMP and/or VLA-4 activity (e.g. cell adhesion) as compared to control indicates that the candidate composition is a modulator of the nitric oxide/cGMP signaling pathway effect on cGMP and/or VLA-4 in a cell extract, cell, tissue, organ, organism or individual. Fluorescence microscopy or a fluorescence imaging can be used to determine the amount of and/or the location of the detectable composition or moiety in a sample cell. The screening, e.g., high-throughput screening, method can comprise high-content imaging on a multi-well plate. The screening can be constructed and practiced on a multi-well plate. (Typically, wells are arranged in two-dimensional linear arrays on the multi-well platform. However, the wells can be provided in any type of array, such as geometric or non-geometric arrays. Commonly used numbers of wells include 24, 96, 384, 864, 1,536, 3,456, and 9,600.) Transmission electron microscopy (TEM) can be used to determine the amount of and/or the location of the detectable composition or moiety in the cell extract, cell, tissue, organ, organism or individual. This technique can be adapted to a plate-reader format for high-throughput screening of drugs that modulate autophagy, i.e., high-throughput detection of autophagic (autophagosome) levels and/or activity in cells or tissues. Compositions disclosed in U.S. Patent Application Document No. 20120042398 (e.g., cadaverine derivatives) can localize into or detect autophagosomes (AV) or AV subpopulations, and these compositions can comprise any detectable moiety or group, e.g., cadaverine derivative(s), or fluorescent-, bioluminescent, radioactive- and/or paramagnetic-conjugated cadaverine reagents.

In addition to the methodologies described herein, for generally applicable methods and materials that can be employed or modified for use in high-throughput format, high-content imaging to examine a cell sample for a nitric oxide/cGMP signaling pathway modulator effect on a cGMP and/or VLA-4-related cell morphology, see e.g. Bova, et al., J. Biomol. Screening, “A label-free approach to identify inhibitors of alpha4-beta7 mediated cell adhesion to MadCAM”, 2011 June; 16(5):536-44. Epub 2011 Mar. 15.

In preferred embodiments, the methods of the invention are conducted in a high-throughput format.

Exemplary high-throughput assay systems include, but are not limited to, an Applied Biosystems plate-reader system (using a plate with any number of wells, including, but not limited to, a 96-well plate, a-384 well plate, a 768-well plate, a 1,536-well plate, a 3,456-well plate, a 6,144-well plate, and a plate with 30,000 or more wells), the ABI 7900 Micro Fluidic Card system (using a card with any number of wells, including, but not limited to, a 384-well card), other microfluidic systems that exploit the use of TaqMan probes (including, but not limited to, systems described in WO 04083443 A1, and published U.S. Patent Application Nos. 2003-0138829 A1 and 2003-0008308 A1), other micro card systems (including, but not limited to, WO04067175 A1, and published U.S. Patent Application Nos. 2004-083443 A1, 2004-0110275 A1, and 2004-0121364 A1), the Invader® system (Third Wave Technologies), the OpenArray™ system (Biotrove), systems including integrated fluidic circuits (Fluidigm), and other assay systems known in the art. In certain embodiments, multiple different labels are used in each multiplex amplification reaction in a high-throughput multiplex amplification assay system such that a large number of different target nucleic acid sequences can be analyzed on a single plate or card. In certain embodiments, a high-throughput multiplex amplification assay system is capable of analyzing most of the genes in a genome on a single plate or card. In certain embodiments, a high-throughput multiplex amplification assay system is capable of analyzing all genes in an entire genome on a single plate or card. In certain embodiments, a high-throughput multiplex amplification assay system is capable of analyzing most of the nucleic acids in a transcriptome on a single plate or card. In certain embodiments, a high-throughput multiplex amplification assay system is capable of analyzing all of the nucleic acids in a transcriptome on a single plate or card.

The method of the present invention of identifying compounds which are useful to inhibit cell adhesion according to the present invention is readily adaptable to high throughput screening, especially when coupled to HyperCyt™, a preferred system, which delivers beads to a flow cytometer from multiwell plates, see Kuckuck et al. (2001), High Throughput Flow Cytometry, Cytometry, 44, pp 83-90 and Jackson et al. (2002), Mixing Small Volumes for Continuous High-Throughput Flow Cytometry: Performance of a Mixing Y and Peristaltic Sample Delivery, Cytometry, 47, pp 183-191, the entire contents and disclosures of which are hereby incorporated by reference, although as discussed hereinabove, a number of alternative flow cytometry approaches may be used.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing information over networks such as the Internet. For example, the components of the system may be interconnected via any suitable means including over a network, e.g. the ELISA plate reader to the processor or computing device. The processor may take the form of a portable processing device that may be carried by an individual user e.g. lap top, and data can be transmitted to or received from any device, such as for example, server, laptop, desktop, PDA, cell phone capable of receiving data, BLACKBERRY™, and the like. In some embodiments of the invention, the system and the processor may be integrated into a single unit. In another example, a wireless device can be used to receive information and forward it to another processor over a telecommunications network, for example, a text or multi-media message.

The functions of the processor need not be carried out on a single processing device. They may, instead be distributed among a plurality of processors, which may be interconnected over a network. Further, the information can be encoded using encryption methods, e.g. SSL, prior to transmitting over a network or remote user. The information required for decoding the captured encoded images taken from test objects may be stored in databases that are accessible to various users over the same or a different network.

In some embodiments, the data is saved to a data storage device and can be accessed through a web site. Authorized users can log onto the web site, upload scanned images, and immediately receive results on their browser. Results can also be stored in a database for future reviews.

In some embodiments, a web-based service may be implemented using standards for interface and data representation, such as SOAP and XML, to enable third parties to connect their information services and software to the data. This approach would enable seamless data request/response flow among diverse platforms and software applications.

The term “compound” is used herein to refer to any specific chemical compound disclosed herein, including its pharmaceutically acceptable salts within context. Within its use in context, the term generally may refer to a single compound, such as a polypeptide or other molecular entity used in the present invention.

In certain non-limiting embodiments, an increase or a decrease in a subject or test sample of the level of measured protein or gene expression or change in a nitric oxide/cGMP signaling pathway modulator effect on a cGMP and/or VLA-4-related cell morphology as compared to a comparable level of measured protein or gene expression or change in a nitric oxide/cGMP signaling pathway modulator effect on a cGMP and/or VLA-4-related cell morphology of a control subject or sample can be an increase or decrease in the magnitude of approximately ±5,000-10,000%, or approximately ±2,500-5,000%, or approximately ±1,000-2,500%, or approximately ±500-1,000%, or approximately ±250-500%, or approximately ±100-250%, or approximately ±50-100%, or approximately ±25-50%, or approximately ±10-25%, or approximately ±10-20%, or approximately ±10-15%, or approximately ±5-10%, or approximately ±1-5%, or approximately ±0.5-1%, or approximately ±0.1-0.5%, or approximately ±0.01-0.1%, or approximately ±0.001-0.01%, or approximately ±0.0001-0.001%.

The values obtained from controls are reference values representing a known health status and the values obtained from test samples or subjects are reference values representing a known disease status. The term “control”, as used herein, can mean a sample of preferably the same source (e.g. blood, serum, tissue etc.) which is obtained from at least one healthy subject to be compared to the sample to be analyzed. In order to receive comparable results the control as well as the sample should be obtained, handled and treated in the same way. In certain examples, the number of healthy individuals used to obtain a control value may be at least one, preferably at least two, more preferably at least five, most preferably at least ten, in particular at least twenty. However, the values may also be obtained from at least one hundred, one thousand or ten thousand individuals.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Standard techniques for growing cells, separating cells, and where relevant, cloning, DNA isolation, amplification and purification, for enzymatic reactions involving DNA ligase, DNA polymerase, and restriction endonucleases as disclosed above can be employed.

In exemplary embodiments of the invention which comprise detecting the presence of antibodies that are reactive to cGMP and/or VLA-4, antibodies are found in a sample from a subject. The antibodies can be detected by an immunoassay wherein an antibody-protein complex is formed. The antibodies are found in the sample of the subject, e.g. serum. The subject is a human and the implicated disease (e.g. multiple sclerosis, ulcerative colitis, Crohn's disease, rheumatoid arthritis, asthma, acute juvenile onset diabetes (Type 1), AIDS dementia, atopic dermatitis, psoriasis, nephritis, retinitis, acute leukocyte-mediated lung injury, transplant rejection, or graft versus host disease) is idiopathic. Healthy individuals have minimal or low VLA-4 levels as defined by experimental protocol and as determined by conventional ELISA or Western blots. Individuals with a VLA-4-related cell adhesion disorder have significant amount of detectable VLA-4 auto-antibodies, at least 10% more anti-VLA-4 auto-antibodies detected over that from a healthy non-VLA-4-related cell adhesion disorder individual or the level obtained for a population of healthy non-a VLA-4-related cell adhesion disorder individuals by conventional ELISA or Western blots as described herein. Moreover the levels of auto-antibodies correspond with the clinical features of the disease condition. Patients in remission after effective treatment have minimal or undetectable anti-VLA-4 auto-antibodies by conventional ELISA or Western blots. As an example, by undetectable amount of anti-VLA-4 auto-antibodies, it means that no visible band is observed in a Western Blot analysis, wherein human serum is diluted 1:100 and used in blot assays described herein. In one embodiment, the amount of anti-VLA-4 auto-antibodies in a healthy non-a VLA-4-related cell adhesion disorder individual or the average amount in a population of healthy non-a VLA-4-related cell adhesion disorder individuals as determined by conventional ELISA or Western blot can be considered as the background, reference or the control level. The collected samples of serum from the healthy non-a VLA-4-related cell adhesion disorder individuals are diluted 1:100 and used in Western blot assays. The intensity of the visible band is quantified by densitometry. The densitometry intensity can be calibrated with a range of known titer of anti-VLA-4 antibodies reacting with a fixed amount of antigen VLA-4. For example, the range of known antibody titer can be 0 .mu.g/ml, 0.5 .mu.g/ml, 1.0 .mu.g/ml, 1.5 .mu.g/ml, 2.0 .mu.g/ml, 2.5 .mu.g/ml, 3.0 .mu.g/ml, 5 .mu.g/ml, 7.5 .mu.g/ml, 10 .mu.g/ml, and 15 .mu.g/ml and the fixed amount of VLA-4 can be 0.5 .mu.g on a blot. By comparing the densitometry intensity of a human sample with the calibration curve, it is possible to estimate the titer of the anti-VLA-4 in the sample. For the data collected for a population of individuals, the average value and one order of standard deviation is computed. Ideally, a population has about 25 healthy non-a VLA-4-related cell adhesion disorder individuals, preferably more. The statistics, the average value and one order of standard deviation can be uploaded to the computer system and data storage media. Patients having at least 10% more than this average amount of anti-VLA-4 auto-antibodies is likely to have a VLA-4-related cell adhesion disorder, especially if the patient is also presents the clinical significant features of the disease. Methodologies that are similar to those described above can be used to evaluate other targets and disorders described herein.

In one embodiment, the auto-antibodies in the sample are reactive against the VLA-4 that has been extracted from mammalian tissues or recombinant mammalian VLA-4, e.g. the human VLA-4. The sample from the subject can be a blood sample. In other embodiments, the sample is a serum or plasma sample. In one embodiment, the auto-antibodies are detected by a serological immunoassay, such as an enzyme-linked immunosorbant assay or a nephelometric immunoassay.

The term “patient” or “subject” refers to an animal, such as a mammal, or a human, in need of treatment or therapy to which compounds according to the present invention are administered in order to treat a condition or disease state associated with a VLA-4-related cell adhesion disorder, for instance, a particular stage of multiple sclerosis, ulcerative colitis, Crohn's disease, rheumatoid arthritis, asthma, acute juvenile onset diabetes (Type 1), AIDS dementia, atopic dermatitis, psoriasis, nephritis, retinitis, acute leukocyte-mediated lung injury, transplant rejection, and graft versus host disease, using compounds according to the present invention.

A “VLA-4-related cell adhesion disorder” includes diseases and conditions resulting from inflammation implicating α₄β₁-integrin-dependent interaction with the VCAM-1 ligand on endothelial cells and having acute and/or chronic clinical exacerbations, e.g. multiple sclerosis (Yednock et al., Nature 356, 63 (1992); Baron et al., J. Exp. Med. 177, 57 (1993)), meningitis, encephalitis, stroke, other cerebral traumas, inflammatory bowel disease including ulcerative colitis and Crohn's disease (Hamann et al., J. Immunol. 152, 3238 (1994)), (Podolsky et al., J. Clin. Invest. 92, 372 (1993)), rheumatoid arthritis (van Dinther-Janssen et al., J. Immunol. 147, 4207 (1991); van Dinther-Janssen et al., Annals Rheumatic Diseases 52, 672 (1993); Elices et al., J. Clin. Invest. 93, 405 (1994); Postigo et al., J. Clin. Invest: 89, 1445 (1992), asthma (Mulligan et al., J. Immunol. 150, 2407 (1993)) and acute juvenile onset diabetes (Type 1) (Yang et al., PNAS 90, 10494 (1993); Burkly et al., Diabetes 43, 529 (1994); Baron et al., J. Clin. Invest. 93, 1700 (1994)), AIDS dementia (Sasseville et al., Am. J. Path. 144, 27 (1994); atherosclerosis (Cybulsky & Gimbrone, Science 251, 788, L1 et al., Arterioscler. Thromb. 13, 197 (1993)), nephritis (Rabb et al., Springer Semin. Immunopathol. 16, 417-25 (1995)), retinitis, atopic dermatitis, psoriasis, myocardial ischemia, acute leukocyte-mediated lung injury such as occurs in adult respiratory distress syndrome, tumor metastasis including bone metastasis, transplant rejection, graft versus host disease, and cancers including melanoma, multiple myeloma, malignant lymphoma, acute and chronic leukemias, pancreatic cancer, neuroblastoma, small cell and non-small cell lung cancer, mesothelioma, colorectal carcinoma, and breast cancer.

Nitric oxide/cGMP signaling pathway modulators are selected from the group consisting of a nitric oxide donor, a nitric oxide-independent activator of soluble guanylyl cyclase, or a cell permeable analog of cGMP.

“Nitric oxide (NO) donors” include, but are not limited to: (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazeniumdiolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF. Other useful nitric-oxide donor drugs are described in Miller, et al., Recent developments in nitric oxide donor drugs, Br J Pharmacol. 2007 June; 151(3): 305-32, the complete contents of which are incorporated by reference herein.

“Nitric oxide-independent activators of soluble guanylyl cyclase” include, but are not limited to, BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin. Certain compounds that activate sGC NO-independently can be characterized as heme-dependent sGC stimulators, such as BAY 41-2272, BAY 41-8543, and BAY 63-2521, and heme-independent sGC activators, such as BAY 58-2667, and HMR-1766. See Evgenov et al., Nature Reviews Drug Discovery 5, 755-768 (September 2006).

“Cell permeable analogs of cGMP” include, but are not limited to, N²,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “body fluid” and “bodily fluid,” used interchangeably herein, refer to a biological sample of liquid from a mammal, e.g., from a human. Such fluids include aqueous fluids such as serum, plasma, lymph fluid, synovial fluid, follicular fluid, seminal fluid, amniotic fluid, milk, whole blood, urine, cerebrospinal fluid, saliva, sputum, tears, perspiration, mucus, tissue culture medium, tissue extracts, and cellular extracts. Particular bodily fluids that are interest in the context of the present invention include serum, plasma, and blood.

The term “effective” is used to describe an amount of a composition used in the treatment of a VLA-4-related cell adhesion disorder which produces the intended effect within the context of its use.

The term “treatment” or “treating” is used to describe an approach for obtaining beneficial or desired results including and preferably clinical results. For purposes of this invention, beneficial or desired clinical results include, but are not limited to, one or more of the following: alleviation of one or more symptoms, diminishment of extent of disease, stabilized (i.e., not worsening) state of disease, preventing or reducing the likelihood of the spread of disease, reducing the likelihood of occurrence or recurrence of disease, decreasing, amelioration of the disease state, remission (whether partial or total), reduction of incidence of disease and/or symptoms, stabilizing (i.e., not worsening) of a VLA-4-related cell adhesion disorder or improvement of, e.g. symptoms associated with multiple sclerosis, ulcerative colitis, Crohn's disease, rheumatoid arthritis, asthma, acute juvenile onset diabetes (Type 1), AIDS dementia, atopic dermatitis, psoriasis, nephritis, retinitis, acute leukocyte-mediated lung injury, transplant rejection, and graft versus host disease or cancer metastasis. The “treatment” of a VLA-4-related cell adhesion disorder may be administered when no symptoms are present, and such treatment (as the definition of “treatment” indicates) improves one or more biological functions and reduces the incidence or likelihood of disease progression or onset. Also encompassed by “treatment” is a reduction of pathological consequences of any aspect of a VLA-4-related cell adhesion disorder or associated disease states or conditions, including reducing pain and inflammation, reducing infections, improving cardiopulmonary function, stabilizing blood glucose levels, enhancing bronchial dilation, suppressing skin cell growth, reducing high blood pressure, preventing cancer metastasis and other problems associated therewith. All secondary conditions or disease states which occur as a consequence of a VLA-4-related cell adhesion disorder may be reduced or ameliorated.

The term “co-administration” or “combination therapy” is used to describe a therapy in which at least two active compositions in effective amounts are used to treat a VLA-4-related cell adhesion disorder or associated disease states or conditions at the same time. Although the term co-administration preferably includes the administration of two active compositions to the patient at the same time, it is not necessary that the compositions be administered to the patient at the same time, although effective amounts of the individual compositions will be present in the patient at the same time.

Methods of treatment and pharmaceutical compositions of the invention can comprise co-administration of anti-cancer agents in addition to nitric oxide/cGMP signaling pathway modulator anti-cancer agents as described herein. These additional anti-cancer agents include, for example, antimetabolites, inhibitors of topoisomerase I and II, alkylating agents and microtubule inhibitors (e.g., taxol). Specific anticancer co-therapies for use in the present invention include, for example, adriamycin aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof, among others. It is noted that in certain embodiments, where drug resistance (including environmental mediated drug resistance) has occurred, this provides a further rational for the co-administration of compounds.

“Additional VLA-4 antagonists” include, but are not limited to, Tysabri® (natalizumab), AN-100226 (Antegren), CDP323, Firategrast, ATL/TV1102, ATL1102, the VLA-4 antagonists identified in Chigaev, et al. The Journal of Biological Chemistry, 286, 5455-5463 (2011) and Semko, et al., Bioorg Med Chem Lett. 2011 Mar. 15; 21(6):1741-3, clafrinast, RBx-7796, and the VLA-4 antagonists disclosed or referenced in U.S. Pat. No. 7,419,666, EP20100185454, and U.S. Patent Application. Document No. 20110305686, pharmaceutically acceptable salts thereof and mixtures thereof.

According to various embodiments, the compounds according to the present invention may be used for treatment or prevention purposes in the form of a pharmaceutical composition. This pharmaceutical composition may comprise one or more of an active ingredient as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intestinal, parenteral, oral, buccal, nasal, intramuscular, transcutaneous, transdermal, intranasal, intraperitoneal, and topical.

The subject or patient may be chosen from, for example, a human, a mammal such as domesticated animal, or other animal. The subject may have one or more of the disease states, conditions or symptoms associated with a VLA-4-related cell adhesion disorder or associated disease states or conditions.

The compounds according to the present invention may be administered in an effective amount to treat or reduce the likelihood of a VLA-4-related cell adhesion disorder or associated disease states or conditions, or any one or more of the symptoms, disease states or conditions associated with a VLA-4-related cell adhesion disorder or associated disease states or conditions. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of a VLA-4-related cell adhesion disorder or associated disease states for a human patient is that which is an effective amount and may range from as little as 100 μg to at least about 500 mg or more, which may be administered in a manner consistent with the delivery of the drug and the disease state or condition to be treated. In the case of oral administration, active is generally administered from one to four times or more daily. Transdermal patches or other topical administration my administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient preferably ranges from about 0.05 mg/kg to about 10 mg/kg, about 0.1 mg/kg to about 7.5 mg/kg, about 0.25 mg/kg to about 6 mg/kg., about 1.25 to about 5.7 mg/kg.

The dose of a compound according to the present invention may be administered at the first signs of the onset of a VLA-4-related cell adhesion disorder or associated disease states or conditions. For example, the dose may be administered for the purpose of reducing pain and inflammation, reducing infections, improving cardiopulmonary function, stabilizing blood glucose levels, enhancing bronchial dilation, suppressing skin cell growth, reducing high blood pressure, preventing cancer metastasis and/or treating or reducing the likelihood of any one or more of the disease states or conditions which become manifest during a VLA-4-related cell adhesion disorder or associated disease states or conditions, including those symptoms and conditions mentioned above. The dose of active ingredient may be administered at the first sign of relevant symptoms prior to diagnosis, but in anticipation of the disease or disorder or in anticipation of decreased bodily function or any one or more of the other symptoms or secondary disease states or conditions associated a VLA-4-related cell adhesion disorder or associated disease states or conditions.

These and other aspects of the invention are described further in the following illustrative examples.

### EXAMPLES

### Summary

Using fluorescent ligand binding to evaluate the integrin activation state on live cells in real-time, we showed that several small molecules, which specifically modulate nitric oxide/cGMP signaling pathway, as well as a cell permeable cGMP analog, can rapidly down-modulate binding of a VLA-4 specific ligand on cells pre-activated through three Gαᵢ-coupled receptors: wild type CXCR4, CXCR2 (IL-8RB), and a non-desensitizing mutant of formyl peptide receptor (FPR AST). Upon signaling, we detected rapid changes in the ligand dissociation rate. The dissociation rate after inside-out integrin de-activation was similar to the rate for resting cells. In a VLA-4/VCAM-1-specific myeloid cell adhesion system, inhibition of the VLA-4 affinity change by nitric oxide had a statistically significant effect on real-time cell aggregation.

We conclude that nitric oxide/cGMP signaling pathway can rapidly down-modulate the affinity state of the VLA-4 binding pocket, especially under the condition of sustained Gαᵢ-coupled GPCR signaling, generated by a non-desensitizing receptor mutant. This suggests a fundamental role of this pathway in de-activation of integrin-dependent cell adhesion.

We found that the addition of a nitric oxide donor can rapidly induce dissociation of the VLA-4 specific ligand after cellular activation by any of three GPCRs (CXCR4, CXCR2, and FPR). The effect of nitric oxide was also mimicked by a NO-independent cGMP-cyclase activator, as well as a cell permeable analog of cGMP. This indicates that the integrin deactivation mechanism is intracellular, and suggests that deactivation is not related to direct s-nitrosylation. We also detected rapid changes in the dissociation rate constant (k_{off}) of the VLA-4 specific ligand. As shown previously, modulation of the k_{off} directly correlates with changes in the VLA-4 ligand binding affinity [14, 17]. Finally, using a VLA-4/VCAM-1 specific cell adhesion system, we showed that treatment of cells with a nitric oxide donor diminished GPCR activated cell adhesion to the level of un-stimulated (untreated) cells. Taken together, our results indicate that the NO/cGMP signaling pathway can actively down-regulate the affinity of the VLA-4 ligand binding pocket. This observation provides a molecular mechanism for the anti-adhesive activity of nitric oxide donors and drugs that modulate cGMP signaling pathway.

### Example 1

### The Effects of Nitric Oxide/cGMP Signaling in Leukocytes

Nitric oxide, generated by nitric oxide synthase, diffuses across the plasma membrane and through the cytoplasm. In leukocytes NO reacts with the active site of guanylyl cyclase (guanylate GC), and stimulates the production of the intracellular mediator cyclic GMP (cGMP). Next, cGMP interacts with the cGMP-dependent protein kinase (PKG), which phosphorylates multiple substrates, and participates in signal propagation. Cyclic nucleotide phosphodiesterases (PDEs, not shown) can rapidly hydrolyze cGMP and terminate signal propagation. The NO/cGMP signaling pathway can be specifically targeted using small molecules. The nitric oxide donor provides an exogenous source of NO. The activator of soluble guanylyl cyclase binds to GC, and induces enzyme activation in the absence of NO. The cell permeable analog of cGMP diffuses across the plasma membrane, and thus, activates cGMP-dependent signaling.

### Small Molecule Probes for Dissecting the Nitric Oxide/cGMP Pathway

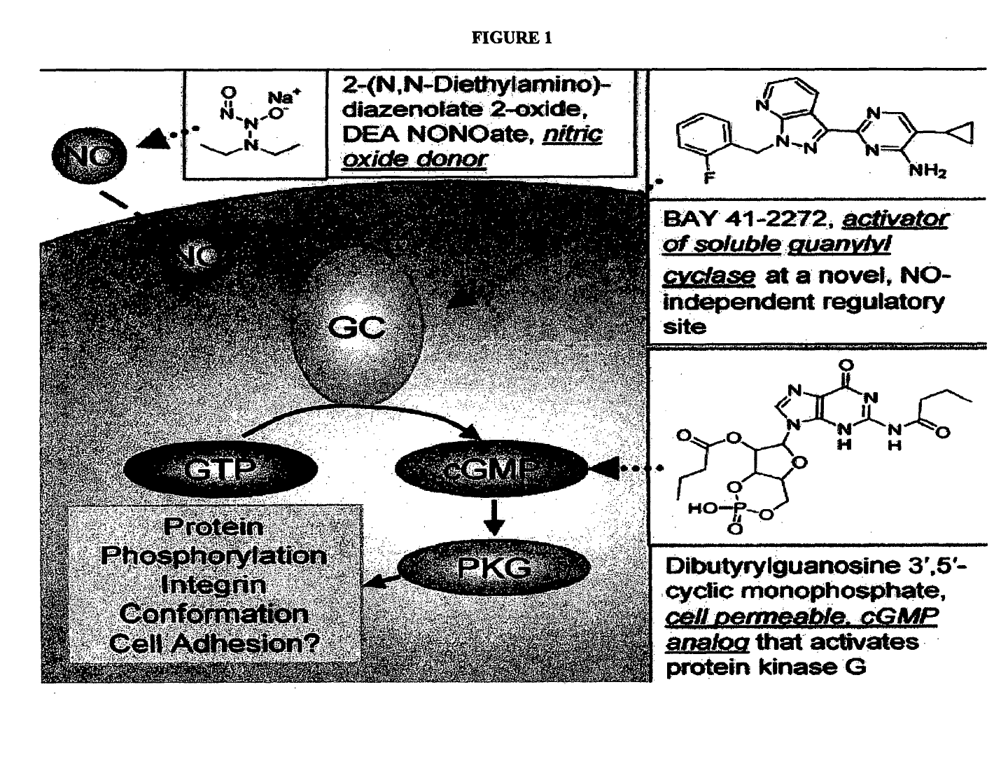

The nitric oxide/cGMP signaling pathway has been described in mature leukocytes, platelets, and hematopoietic progenitors. It is composed of soluble guanylyl cyclase (GC) that serves as an intracellular receptor for nitric oxide (FIG. 1). Upon binding to NO-sensitive guanylyl cyclase, nitric oxide induces a conformational change resulting in the activation of the enzyme [36], and conversion of GTP to cGMP. Cyclic guanosine monophosphate binding leads to the subsequent activation of the cGMP dependent kinase PKG that phosphorylates multiple substrates, and participates in the regulation of platelet adhesion and aggregation [37].

To study the effects of nitric oxide/cGMP signaling in leukocytes, we selected three small molecules that specifically target this pathway (FIG. 1). Diethylamine NONOate can be described as a complex of diethylamine with nitric oxide. It is unstable in aqueous solution and used as nitric oxide donor [38]. BAY 41-2272 is an activator of soluble guanylyl cyclase, which stimulates cGMP production through an NO-independent mechanism [39, 40]. N²,2′-O-dibutyrylguanosine 3′,5′-cyclic monophosphate is a cell permeable cGMP analog that activates protein kinase G [41]. These molecules are shown to stimulate the three initial consecutive steps of the pathway (FIG. 1), and therefore, can be used to mimic NO-dependent signaling.

### Example 2

### Nitric Oxide Donor Induces Rapid Decrease in the Binding of VLA-4 Specific Ligand

### Materials and Methods

Experiments were conducted as described under “Methods”, infra. A, LDV-FITC probe binding and dissociation on U937 cells stably transfected with the non-desensitizing mutant of FPR (ΔST) [48] receptor plotted as mean channel fluorescence (MCF) versus time.

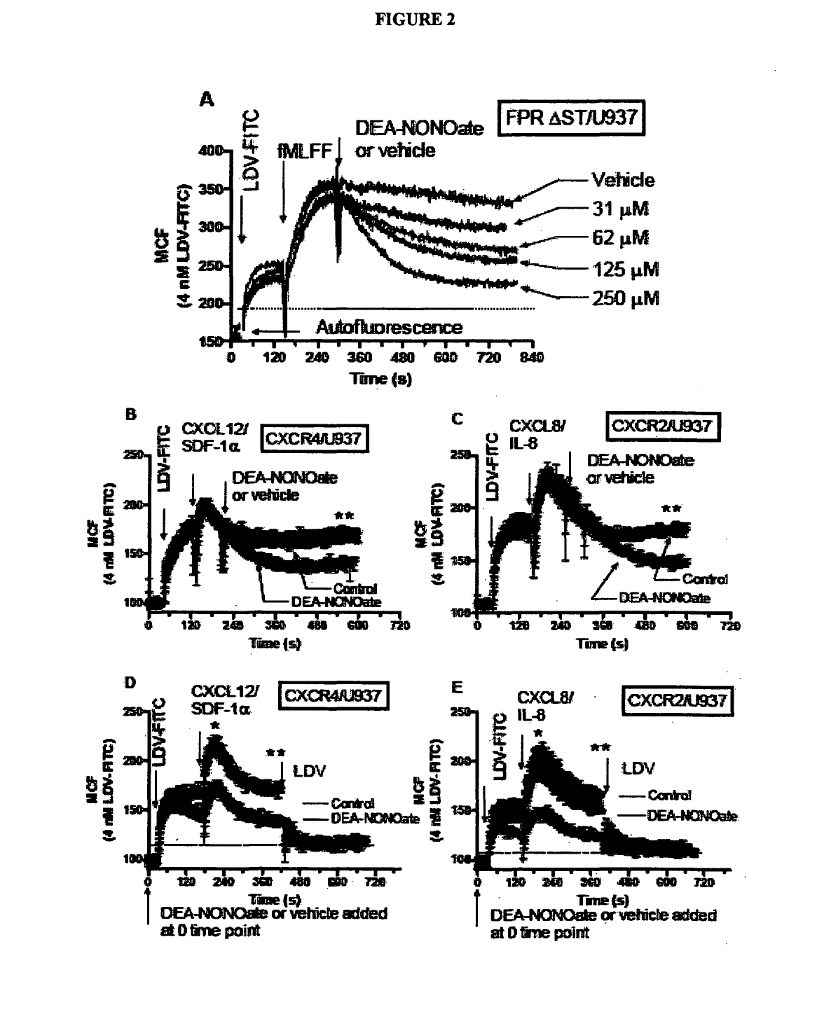

The experiment involved sequential addition of fluorescent LDV-FITC probe (4 nM, below saturation, added 2 min prior to addition of Gαᵢ-coupled receptor ligand, fMLFF, 100 nM), and different concentrations of DEA-NONOate (nitric oxide donor) (arrows). Control cells were treated with vehicle. The MCF value corresponding to cell autofluorescence is indicated by the horizontal arrow. Dashed line indicates the non-specific binding of the LDV-FITC probe determined using an excess of unlabelled LDV competitor (as shown in FIG. 2D,E). Curves are means of two independent determinations calculated on a point-by-point basis (n=2). B, LDV-FITC probe binding and dissociation on U937 cells stably transfected with wild type CXCR4 receptor plotted as mean channel fluorescence (MCF) versus time.

The experiment involved sequential addition of fluorescent LDV-FITC probe (4 nM), CXCL12/SDF-1 (12 nM), and DEA-NONOate (250 μM, nitric oxide donor) or vehicle (control) (arrows). Rapid and reversible binding of the probe reflects the VLA-4 affinity change [14]. Curves are means of two independent determinations calculated on a point-by-point basis (n=2). SEM of mean, calculated on a point by point basis, indicated using error bars to show significance of the difference between treatment and control samples. C, LDV-FITC probe binding and dissociation on U937 cells stably transfected with wild type CXCR2/IL-8RB receptor plotted as mean channel fluorescence (MCF) versus time.

The experiment involved sequential addition of the fluorescent LDV-FITC probe (4 nM), CXCL8/IL-8 (20 nM), and DEA-NONOate (250 μM, nitric oxide donor) or vehicle (control) (arrows). This experiment is analogous to the one shown in panel B. One representative experiment of three experiments is shown. Curves are means of two independent determinations calculated on a point-by-point basis (n=2). D, LDV-FITC probe binding and dissociation on U937 cells stably transfected with wild type CXCR4 receptor plotted as mean channel fluorescence (MCF) versus time.

The experiment involved sequential addition of the DEA-NONOate (250 μM, nitric oxide donor) or vehicle (control) at the 0 time point, and the fluorescent LDV-FITC probe (4 nM), CXCL12/SDF-1 (12 nM) (arrows). Rapid and reversible binding of the probe reflects the VLA-4 affinity change [14]. Excess unlabelled competitor LDV (1 μM) is added at the end of the experiment to determine the non-specific binding of the probe. Curves are means of two independent determinations calculated on a point-by-point basis (n=2). SEM, calculated on a point by point basis, is indicated using error bars to show the significance of the difference between treatment and control samples. E, LDV-FITC probe binding and dissociation on U937 cells stably transfected with wild type CXCR2/IL-8RB receptor plotted as mean channel fluorescence (MCF) versus time.

The experiment involved sequential addition of DEA-NONOate (250 μM, nitric oxide donor) or vehicle (control) at the 0 time point, and the fluorescent LDV-FITC probe (4 nM), CXCL8/IL-8 (20 nM) (arrows). Excess unlabelled competitor LDV (1 μM) added at the end of the experiment to determine the non-specific binding of the probe. This experiment is analogous to the one shown in panel D. One representative experiment of three experiments is shown. Curves are means of two independent determinations calculated on a point-by-point basis (n=2). According to the unpaired t test, the means are significantly different (p<0.05) at the peak of activation (marked on panels D and E as “*”), and at the steady state (marked on panels B-E as “**”). Experiments shown in the different panels were performed using different instruments, and therefore MCF values are not identical.

### Nitric Oxide Donor Induces Rapid Decrease in the Binding of VLA-4 Specific Ligand

Previously, we described and characterized in detail a model ligand an LDV-FITC containing small molecule ([14, 42-44], and references therein) for the detection of VLA-4 conformational regulation. This VLA-4 specific fluorescent probe was based on a highly specific α₄β₁-integrin inhibitor BIO1211, which contains the Leu-Asp-Val (LDV) ligand binding motif from the alternatively spliced connecting segment-1 (CS-1) peptide of cellular fibronectin [17, 45]. We established that integrin affinity changes, detected using this probe, vary in parallel with the natural VLA-4 ligand, human VCAM-1 [46]. For real-time detection of rapid integrin conformational changes, cells were treated with LDV-FITC (FIG. 2, first arrow), which was added after establishing a baseline for unstained cells, indicated on FIG. 2A as “autofluorescence”. Next, data were acquired for 2-3 minutes, and cells were activated with fMLFF (high affinity FPR ligand), CXCL12/SDF-1 (CXCR4 ligand), or CXCL8/IL-8 (CXCR2 ligand), for FPR, CXCR4, CXCR2 transfected cells, respectively (FIGS. 2A, B, and C). The concentration of the LDV-FITC probe used in the experiments (4 nM) was below the dissociation constant (K_{d}) for its binding to resting VLA-4 (low affinity state, K_{d}˜12 nM), and above the K_{d} for physiologically activated VLA-4 (high affinity state, K_{d}˜1-2 nM) [14]. Therefore, the transition from the low affinity to the high affinity receptor state led to increased binding of the probe (from ˜25% to ˜70-80% of receptor occupancy, as calculated based on the one site binding equation). The change in occupancy was detected as a rapid increase in the mean channel fluorescence (MCF). This signal increase was sustained for the case of a non-desensitizing mutant of FRP (FIG. 2A), and reversible for the wild-type receptors (CXCR4, and CXCR2, FIG. 2B,C). Next, cells were treated with the nitric oxide donor, or vehicle (control). Acquisition was re-established, and data were acquired continuously for up to 720-840 s. Addition of the nitric oxide donor resulted in a rapid and dose-dependent decrease in the binding of the VLA-4 specific ligand. In the absence of receptor desensitization, the effect of nitric oxide was more evident in cells transfected with a non-desensitizing mutant of FPR (vehicle, FIG. 2A) [47, 48]. However, the effect of the nitric oxide donor was statistically significant for both wild-type GPCRs. A faster and more pronounced signal decrease was detected (see black lines in FIG. 2B, 2C). To emphasize statistically the difference between control and experimental samples, standard errors of mean are indicated using error bars for every experimental point in FIG. 2B, 2C, 2D, 2E.

Next, we studied the effect of nitric oxide donor added prior to cell activation. DEA-NONOate was added at the 0 time point as indicated by the arrow (FIG. 2D, 2E). This resulted in a significant decrease in the magnitude of the response for both SDF-1 and IL-8 treated cells. Moreover, the effect of nitric oxide can be detected prior to cell activation. This suggests that at rest a small number of VLA-4 molecules exist in the activated conformation, and addition of nitric oxide donor deactivates these integrins. It worth noting that the nonspecific binding of the LDV-FITC probe remained identical for both control and treated samples (compare sample fluorescence after addition of LDV). Thus, the nitric oxide donor rapidly decreased binding of the VLA-4 specific fluorescent ligand after cell activation through three Gαᵢ-coupled GPCRs. Pretreatment with the nitric oxide donor significantly diminished the magnitude of the response.

### Example 3

### Activator of Soluble Guanylyl Cyclase Induces a Dose-Dependent Decrease in the Binding of the VLA-4 Specific Ligand

### Materials and Methods

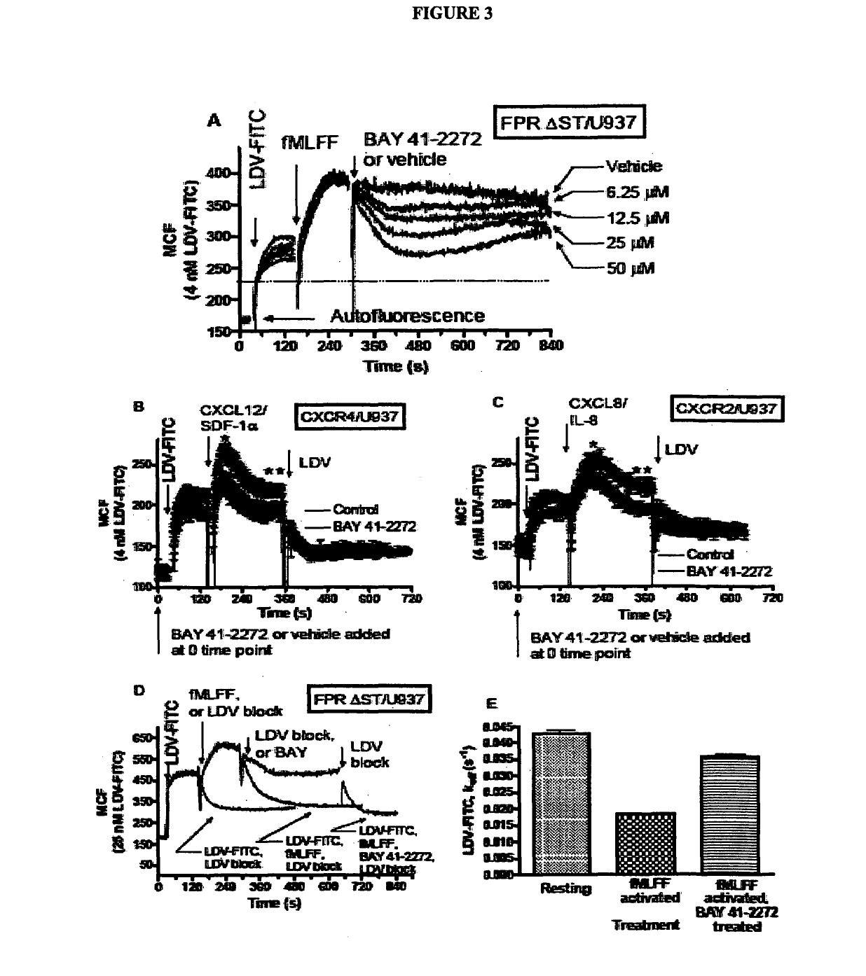

Experiments were conducted as described under “Methods”, infra. A, LDV-FITC probe binding and dissociation on U937 cells stably transfected with the non-desensitizing mutant of FPR plotted as mean channel fluorescence (MCF) versus time. The experiment involved sequential addition of the fluorescent LDV-FITC probe (4 nM, below saturation, added 2 min prior to addition of the Gαᵢ-coupled receptor ligand, fMLFF, 100 nM), and different concentrations of BAY 41-2272 (guanylyl cyclase activator) (arrows). Control cells were treated with vehicle. The MCF value corresponding to cell autofluorescence is indicated by the horizontal arrow. Dashed line indicates the non-specific binding of the LDV-FITC probe determined using excess unlabelled LDV competitor (as shown on FIG. 3B). Rapid and reversible binding of the probe reflects the VLA-4 affinity change [14]. Curves are means of two independent runs calculated on a point-by-point basis (n=2). B, LDV-FITC probe binding and dissociation on U937 cells stably transfected with wild type CXCR4 receptor plotted as mean channel fluorescence (MCF) versus time.

The experiment involved sequential addition of the BAY 41-2272 (100 μM, guanylyl cyclase activator) or vehicle (control) at the 0 time point, and the fluorescent LDV-FITC probe (4 nM), and CXCL12/SDF-1 (12 nM) (arrows). Rapid and reversible binding of the probe reflects the VLA-4 affinity change [14]. Excess unlabelled competitor LDV (1 μM) is added at the end of the experiment to determine the non-specific binding of the probe. Curves are means of three independent determinations calculated on a point-by-point basis (n=3). SEM, calculated on a point by point basis, is indicated using error bars to show the significance of the difference between treatment and control samples. C, LDV-FITC probe binding and dissociation on U937 cells stably transfected with wild type CXCR2/IL-8RB receptor plotted as mean channel fluorescence (MCF) versus time.

The experiment involved sequential addition of BAY 41-2272 (100 μM, guanylyl cyclase activator) or vehicle (control) at the 0 time point, and the fluorescent LDV-FITC probe (4 nM), and CXCL8/IL-8 (20 nM) (arrows). Excess unlabelled competitor LDV (1 μM) added at the end of the experiment to determine the non-specific binding of the probe. This experiment is analogous to the one shown in panel B. One representative experiment of two experiments is shown. Curves are means of two independent determinations calculated on a point-by-point basis (n=2). According to the unpaired t test, the means are significantly different (p<0.05) at the peak of activation (marked on panels B and C as “*”), and at the steady state (marked in panels B and C as “**”). D, Kinetic analysis of binding and dissociation of LDV-FITC probe on U937 cells stably transfected with the non-desensitizing mutant of FPR. Cells were sequentially treated with the LDV-FITC probe (25 nM, near saturation), the Gαᵢ-coupled receptor ligand (fMLFF, 100 nM), BAY 41-2272 (guanylyl cyclase activator, 50 μM) (arrows). At time points indicated by arrows, cells were treated with excess unlabeled LDV containing small molecule (2 μM), and the dissociation of the fluorescent molecule was followed. Dissociation rate constants (k_{off}) were obtained by fitting dissociation curves to a single exponential decay equation (as described in the text). Experiments shown in the different panels were performed using different instruments, and therefore MCF values are not identical. E, Dissociation rate values, obtained in experiments analogous to panel B, summarized as a bar graph showing mean and SEM (n=4). Colors of the dissociation curves in panel D and bars on panel E are matching. The difference between k_{off}s for “resting” and “fMLFF activated”, and between “fMLFF activated” and “fMLFF activated and treated with BAY 41-2272” is statistically significant (P=0.0006<0.05) as calculated by one-way analysis of variance (ANOVA) using GraphPad Prism software.

### Activator of Soluble Guanylyl Cyclase Induces a Dose-Dependent Decrease in the Binding of the VLA-4 Specific Ligand

To confirm that the effect of nitric oxide can be mimicked using a nitric oxide-independent activator of soluble guanylyl cyclase, we repeated the experiments shown in FIG. 2A using BAY 41-2272 (FIG. 3A). Cells, transfected with a non-desensitizing mutant of FPR, were sequentially treated with LDV-FITC (4 nM), fMLFF, vehicle, or indicated concentrations of the soluble guanylyl cyclase activator. We observed a significant decrease in LDV-FITC binding, comparable to the effect induced by the nitric oxide donor (FIG. 2A). However, the decrease in LDV-FITC binding was partially reversible. This phenomenon can be rationalized, in terms of the proposed feedback loops that regulate cGMP production. Intracellular cGMP can directly stimulate the catalytic activity of several cyclic nucleotide phosphodiesterases (PDEs) that hydrolyze cGMP [49-51]. Another possibility is activation of PDEs through phosphorylation by cGMP-dependent protein kinase (PKG) (FIG. 1) [50-53].

Next, we studied the effect of the nitric oxide-independent activator of soluble guanylyl cyclase added prior to cell activation. BAY 41-2272 was added at the 0 time point as indicated by the arrow (FIG. 3B, 3C). This resulted in a decrease in the magnitude of the response for both SDF-1 and IL-8 treated cells in a manner comparable to the effect of nitric oxide donor. Similarly, the effect of activator of soluble guanylyl cyclase can be detected prior to cell activation. Thus, the nitric oxide-independent activator of soluble guanylyl cyclase induces a dose-dependent decrease in binding of the VLA-4 specific ligand, and pretreatment with the activator of soluble guanylyl cyclase significantly diminished the magnitude of the response after activation.

### Dissociation Rate Analysis Revealed Rapid Changes in the Dissociation Rate of the VLA-4 Specific Ligand

As shown previously, for different states of VLA-4 affinity, the LDV-FITC equilibrium dissociation constant K_{d} varied inversely with the dissociation rate constant (k_{off}). This implies that the ligand association rate constant is essentially independent of receptor conformation (for example see Table I in [17]), or Table I in [14]). Therefore, the dissociation rate analysis can be used to assess the affinity state of the VLA-4 integrin binding pocket.

To saturate the majority of low affinity sites, cells transfected with a non-desensitizing mutant of FPR were preincubated with a higher concentration of the VLA-4 specific ligand (25 nM). Since the K_{d} for the low affinity state is ˜12 nM (Table I in [14]), at 25 nM˜70% of sites are occupied before activation. Next, an excess of the unlabeled LDV competitor (labeled on FIG. 3D as “LDV block”) is added to induce dissociation of the LDV-FITC probe. After activation by fMLFF, because of the rapid affinity change, little additional binding of the probe was seen (FIG. 3D, green and red lines). Addition of the nitric oxide-independent activator of the soluble guanylyl cyclase returned the binding of the probe to a level similar to the binding before fMLFF addition.

Next, the regions of the ligand-binding curves corresponding to the dissociation of the LDV-FITC probe were fitted to a single exponential decay equation. The resulting dissociation rate constants (k_{off}, s⁻¹) are shown graphically in FIG. 3E. At rest, the majority of the VLA-4 molecules exhibit rapid probe dissociation, corresponding to the low affinity state of the ligand binding pocket (FIG. 3D, 3E, blue curve “LDV-FITC, LDV block”, k_{off}˜0.04±0.001 s⁻¹). After cell activation by fMLFF, the dissociation rate was significantly slower (FIG. 3 D, 3E, red curve “LDV-FITC, fMLFF, LDV block”, k_{off}˜0.018±0.0001 s⁻¹). The slower k_{off} corresponds to higher ligand binding affinity [14, 17, 46]. After the addition of the nitric oxide-independent activator of soluble guanylyl cyclase, dissociation rates were comparable to the rate for the resting state (FIG. 3 D, 3E, green curve “LDV-FITC, fMLFF, BAY 41-2272, LDV block”, k_{off}˜0.036±0.0007 s⁻¹). This suggests that activation of guanylyl cyclase can actively down-regulate the affinity state of the VLA-4 integrin ligand binding pocket, even under the condition with the continuously signaling non-desensitizing GPCR mutant. The affinity state induced by guanylyl cyclase activator was quantitatively similar to the resting state before activation. The resting VLA-4 conformation on U937 cells exhibits the lowest physiological affinity. It is worth noting, that this result is comparable to the effect of Gas-coupled GPCRs on VLA-4 conformation (compare FIG. 3D in the current manuscript and FIG. 2C, 2D in [21]). This result is especially interesting in light of the structural relationship of the two second messengers cAMP and cGMP, originating from these signaling pathways.

### Example 4

### Dibutyrylguanosine 3′,5′-Cyclic Monophosphate Induces Rapid and Reversible Changes in the Binding of the VLA-4 Specific Ligand

### Materials and Methods

LDV-FITC probe binding and dissociation on U937 cells stably transfected with the non-desensitizing mutant of FPR plotted as mean channel fluorescence (MCF) versus time. The experiment involved sequential addition of the fluorescent LDV-FITC probe (4 nM, below saturation, added 2 min prior to addition of the Gαᵢ-coupled receptor ligand, fMLFF, 100 nM), and different concentrations of dibutyrylguanosine 3′,5′-cyclic monophosphate (cell permeable cGMP analog) (arrows). Control cells were treated with vehicle. The MCF value corresponding to cell autofluorescence is indicated by the horizontal arrow. Dashed line indicates the non-specific binding of the LDV-FITC probe determined using excess unlabelled LDV competitor (as shown on FIG. 3B). Rapid and reversible binding of the probe reflects the VLA-4 affinity change [14]. Curves are means out of two independent determinations calculated on a point-by-point basis (n=2).

LDV-FITC probe binding and dissociation on U937 cells stably transfected with the non-desensitizing mutant of FPR plotted as mean channel fluorescence (MCF) versus time. The experiment involved sequential addition of the fluorescent LDV-FITC probe (4 nM, below saturation, added 2 min prior to addition of the Gαᵢ-coupled receptor ligand, fMLFF, 100 nM), and different concentrations of dibutyrylguanosine 3′,5′-cyclic monophosphate (cell permeable cGMP analog) (arrows). Control cells were treated with vehicle. The MCF value corresponding to cell autofluorescence is indicated by the horizontal arrow. Dashed line indicates the non-specific binding of the LDV-FITC probe determined using excess unlabelled LDV competitor (as shown on FIG. 3B). Rapid and reversible binding of the probe reflects the VLA-4 affinity change [14]. Curves are means out of two independent determinations calculated on a point-by-point basis (n=2).

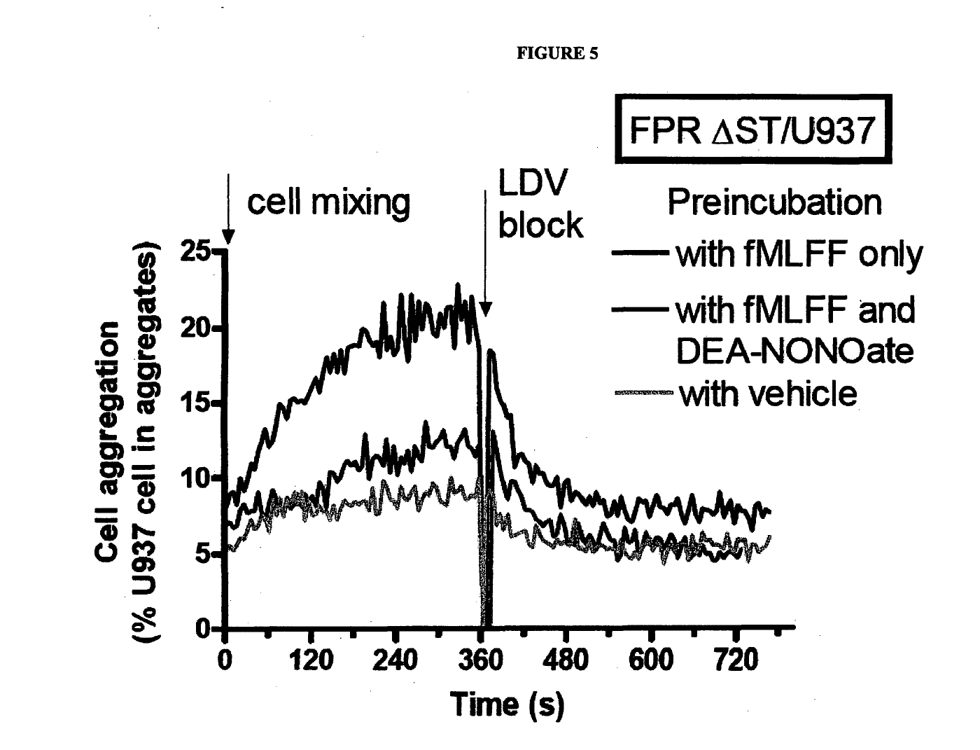

Real-time aggregation experiments were conducted as described under “Methods”, infra. U937/AST FPR stably transfected cells, which constitutively express VLA-4, were labeled with red fluorescent dye, and B78H1/VCAM-1 transfectants were stained with green fluorescent dye. Labeled cells were preincubated for 10 min at 37° C. with fMLFF only (100 nM, activated control), DMSO (vehicle, resting cells control), or with fMLFF and DEA-NONOate (250 μM, nitric oxide donor) in a manner analogous to the experiment showed in FIG. 2A. Next, cells were mixed and real-time cell aggregation (red and green double positive events) was followed. To determine the level of VLA-4 dependent cell aggregation, 6 min after cell mixing, excess unlabelled VLA-4 specific ligand was added (arrow, LDV block, 2 μM). This induced rapid cellular disaggregation to the level of non-specific binding. A representative experiment out of three experiments is shown in FIG. 5.

The Effect of the Cell Permeable Analog of cGMP on Real-Time Binding of the LDV-FITC Probe

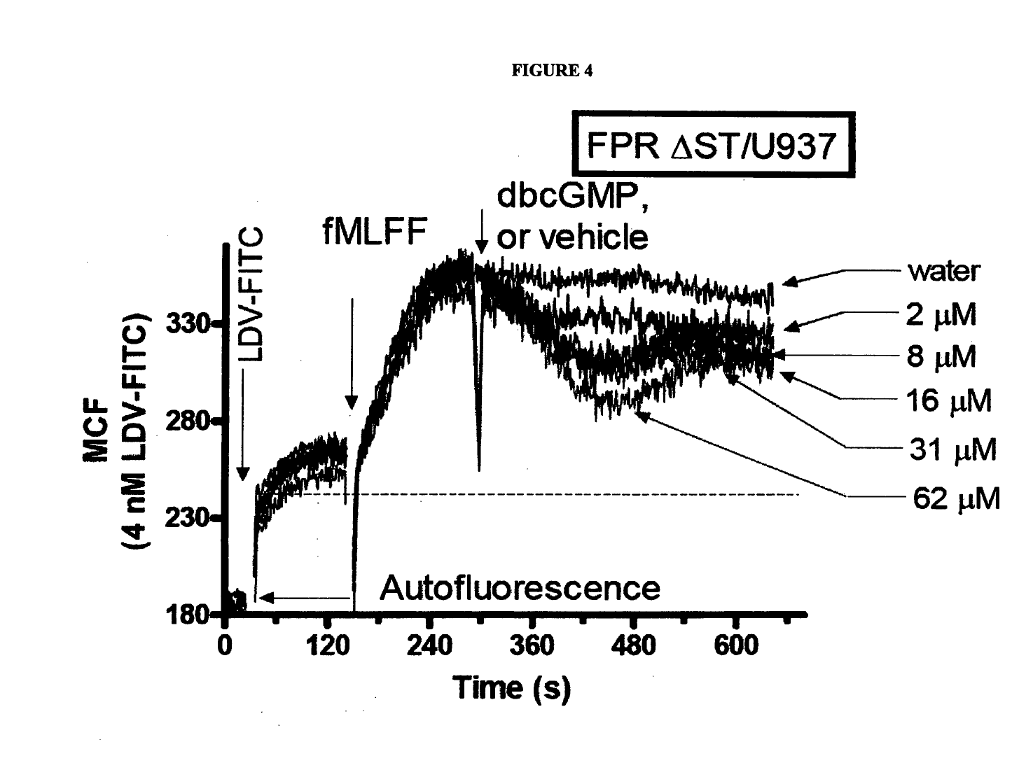

We studied the effect of the cell permeable analog of cGMP on real-time binding of the LDV-FITC probe (FIG. 4). Addition of dbcGMP induced a dose-dependent decrease in the binding of the probe. However, the effect of dbcGMP was reversible. These kinetics are compatible with negative feedback loops that regulate cGMP dependent signaling. Activation of PDEs directly by cGMP binding, or indirectly after being phosphorylated by a cGMP dependent kinase (PKG), has been previously reported [49-53].

Thus, all three probes specifically targeting the NO/cGMP pathway (the nitric oxide donor, the nitric oxide-independent activator of soluble guanylyl cyclase, and the cell permeable analog of cGMP) were found to decrease binding of the VLA-4 specific ligand, with similar kinetics, after cell activation through Gαᵢ-coupled GPCRs. To study the effects of NO/cGMP signaling on cell aggregation, we used a model system, consisting of U937 cells, stably transfected with GPCR in the experiments described above (FIGS. 2, 3, 4), and a mouse melanoma cell line stably transfected with human VCAM-1. The unlabelled VLA-4 specific ligand (LDV), analogous to the LDV-FITC probe, was used to identify VLA-4/VCAM-1 specific cell aggregation. This model system has been described and characterized previously [42, 46, 54, 55].

### The Effect of Nitric Oxide/cGMP Signaling Pathway Activation on VLA-4-VCAM-1 Dependent Cell Adhesion

Prior to the experiment, individual cell populations were stained with either of two fluorescent dyes (red and green). Next, the cell populations were mixed, and the appearance of double positive events, representing cellular aggregates, was followed in real-time by flow cytometry (see FIG. 1, 2, 3 in [55] for method details). Because nitric oxide represents a “natural” signaling molecule, and the effect of nitric oxide was not reversible during the first several hundred seconds after treatment (FIG. 2A), for aggregation experiments cell were treated with the NO-donor (FIG. 5).

Resting (unstimulated) cells showed a very small increase in the % U937 cells in the cell aggregate (FIG. 5, light gray line, labeled “with vehicle”). Inside-out activation resulted in a rapid increase in cell aggregation during the first six minutes after mixing the cell populations (FIG. 5, black line, labeled “with fMLFF only”). Addition of the unlabelled VLA-4 specific ligand “LDV block” resulted in rapid cellular disaggregation, indicating that the majority of aggregates were VLA-4 dependent. The overall extent of activated cell aggregation was similar to previously published data [46]. Pretreatment of U937 cells with fMLFF, and subsequently with nitric oxide donor, in a manner similar to the FIG. 2A, abolished fMLFF-dependent cellular aggregation (gray line, labeled “with fMLFF and DEA-NONOate”). In fact, cell aggregation in this experiment was very similar to the aggregation of the resting cell (untreated control).

Thus, treatment of activated cells with NO-donor only abolished the effect of GPCR-dependent cell activation, and did not affect resting cell aggregation. This result is additionally supported by the LDV-FITC ligand binding kinetics data (FIG. 3B, 3C). Activation of guanylyl cyclase induced a rapid decrease of the VLA-4 ligand binding affinity to a level that was quantitatively similar to the resting state.

The NO/cGMP signaling pathway therefore provides an antagonistic signal that can rapidly and actively decrease the affinity state of the VLA-4 ligand binding pocket, and this results in the modulation of VLA-4/VCAM-1 dependent cellular aggregation.

### Discussion of Experimental Results

### Inside-Out Deactivation of Integrins

A current paradigm of the inside-out activation of integrins implies an instantaneous triggering of integrin conformational changes, where a chemokine signal appears to be closely opposed to the integrin [56]. An “updated” adhesion cascade includes several steps in addition to the traditional tethering, rolling, and arrest [57]. While integrin adhesion research is largely focused on activating pathways, the inhibitory Gas-coupled GPCR/cAMP-dependent signaling pathways is acknowledged for platelet regulation [58]. The relative lack of interest in the integrin deactivation pathways is potentially compensated by the identification of antagonists that competitively block adhesive interactions, and thus, provide a desirable therapeutic effect [59].

However, it is arguable that deactivation of the signaling pathway is as appealing as a direct blockade of the activating signaling using receptor antagonists. It was established, that in order to induce a half-optimal elevation of the signal in leukocytes, only a very small fraction of occupied cellular receptors is required. In some cases, this fraction may be less than 0.1% of the total number of receptors [60]. This is dependent on significant signal amplification for both stimulatory and inhibitory pathways [61]. Therefore, from a therapeutic point of view, it would be very difficult to completely block the occupancy of activating chemokine receptors using receptor-specific antagonists. A small fraction of activating receptors occupied by the ligand, may be sufficient to trigger the adhesion signal. A plausible scenario would be to take advantage of natural regulatory pathways to counteract unwanted signaling, especially because antagonistic pathways potentially have similar amplification capacity [60, 61].

### NO-Dependent VLA-4 Deactivation and Hematopoietic Stem Cell Mobilization

The VLA-4 integrin is critical for the interaction of hematopoietic progenitors and stromal cells [2,3]. Blocking of the VLA-4/VCAM-1 interaction using anti-VLA-4 antibodies, small molecule competitive as well as allosteric VLA-4 antagonists, results in the mobilization of progenitors into the peripheral blood [28-32, 62]. Endothelial nitric oxide synthase (eNOS), one of the major enzymes, producing nitric oxide in the vasculature, is essential for the mobilization of stem and progenitor cells from the bone marrow stem cell niche. Mice lacking eNOS showed a defect in progenitor mobilization [26]. Nitric oxide synthase-derived nitric oxide regulates the bone marrow environment, and is envisioned as a direct mediator of cell mobilization [27]. Our current finding that nitric oxide/cGMP signaling pathway can actively down-regulate VLA-4 affinity, even under conditions of constant signaling, induced by a non-desensitizing mutant of GPCR, indicates that VLA-4 conformational deactivation provides a plausible explanation for the molecular basics of nitric oxide signaling-induced progenitor mobilization.

### Conclusions

We conclude that the nitric oxide/cGMP signalling pathway dramatically decreases the up-regulation of VLA-4 integrin ligand-binding affinity, when triggered prior to inside-out integrin activation, and rapidly down-modulates VLA-4 affinity, when induced after integrin activation. This conformational change results in a significant down-regulation of VLA-4-dependent cell adhesion, suggesting a major role of this pathway in the regulation of inside-out integrin de-activation and cell de-adhesion (mobilization).

### Methods

### Materials

The VLA-4 specific ligand [14, 46, 47] 4-((N′-2-methylphenyl)ureido)-phenylacetyl-L-leucyl-L-aspartyl-L-valyl-L-prolyl-L-alanyl-L-alanyl-L-lysine (LDV containing small molecule), and its FITC-conjugated analog (LDV-FITC) were synthesized at Commonwealth Biotechnologies. Human recombinant CXCL12/SDF-1α, and recombinant human CXCL8/IL-8 were from R&D Systems. All other reagents were from Sigma-Aldrich. Stock solutions were prepared in DMSO, at concentrations˜1000 fold higher than the final concentration. Usually, 1 μl of stock solution was added to 1 ml of cell suspension yielding a final DMSO concentration of 0.1%. Control samples were treated with an equal amount of pure DMSO (vehicle). CXCL12/SDF-1α and CXCL8/IL-8 solutions were prepared using water, and used according to manufacturer's instructions.

### Cell Lines and Transfectant Construct

The human histiocytic lymphoma cell line U937 and mouse melanoma cell line B78H1 were purchased from ATCC. Wild type CXCR4 (CD184) receptor, and CXCR2, IL-8RB, (CD128b, CD182) stably transfected U937 cells, and site-directed mutants of the FPR (non-desensitizing mutant of FPR ΔST) in U937 cells were prepared as described [73] and were a gift of Dr. Eric Prossnitz (University of New Mexico). For transfection of B78H1 cells, full-length human VCAM-1 cDNA was a kind gift from Dr. Roy Lobb of Biogen Inc. The original construct [74] was subcloned into the pTRACER vector (Invitrogen). Transfection into B78H1 was done using the LipofectAMINE Reagent (Invitrogen). High expressors were selected using the MoFlo Flow Cytometer (DakoCytomation). Cells were grown in RPMI 1640 (supplemented with 2 mm 1-glutamine, 100 units/ml penicillin, 100 g/ml streptomycin, 10 mm HEPES, pH 7.4, and 10% heat-inactivated fetal bovine serum) and then harvested and resuspended in 1 ml of HEPES buffer (110 mM NaCl, 10 mM KCl, 10 mM glucose, 1 mM MgCl₂, 1.5 mM CaCl₂, and 30 mm HEPES, pH 7.4) containing 0.1% human serum albumin and stored on ice. The buffer was depleted of lipopolysaccharide by affinity chromatography over polymyxin B sepharose (Detoxigel; Pierce Scientific). Cells were counted using the Coulter Multisizer/Z2 analyzer (Beckman Coulter). For experiments, cells were suspended in the same HEPES buffer at 1×10⁶ cells/ml and warmed to 37° C. Alternatively, cells were resuspended in warm RPMI (37° C.) and used immediately.

### Kinetic Analysis of Binding and Dissociation of VLA-4 Specific Ligand

Kinetic analysis of the binding and dissociation of the LDV-FITC probe was described previously [14, 46]. Briefly, cells (1×10⁶ cells/ml) were preincubated in HEPES buffer containing 0.1% HSA or RPMI under different incubating conditions for 10-20 min at 37° C. Flow cytometric data were acquired for up to 1024 s at 37° C. while the samples were stirred continuously at 300 rpm with a 5×2 mm magnetic stir bar (Bel-Art Products). For real-time affinity activation experiments, 4 nM LDV-FITC was added after establishing a baseline for unstained cells marked on figures as “autofluorescence”. Next, different ligands were added and acquisition was re-established, creating a 5-10 s gap in the time course. For activation, cells were treated with different GPCR ligands at saturating concentration (10 times or higher than K_{d}). In several experiments cells were treated sequentially with two different compounds. Acquisition was re-established, and data were acquired continuously for up to 1024 s. The concentration of the LDV-FITC probe used in the experiments (4 nM) was below the dissociation constant (K_{d}) for its binding to resting VLA-4 (low affinity state, K_{d}˜12 nM), and above the K_{d} for physiologically activated VLA-4 (high affinity state, K_{d}˜1-2 nM) [14]. Therefore, the transition from the low affinity to the high affinity receptor state led to increased binding of the probe (from ˜25% to ˜70-80% of receptor occupancy, as calculated based on the one site binding equation), which was detected as an increase in the mean channel fluorescence (MCF). For kinetic dissociation measurements, cell samples were preincubated with the fluorescent probe (25 nM), treated with excess unlabeled LDV containing small molecule (2 μM) and the dissociation of the fluorescent molecule was followed. The resulting data were converted to MCF versus time using FCSQuery software developed by Dr. Bruce Edwards (University of New Mexico).

### Cell Adhesion Assay

The cell suspension adhesion assay has been described previously [46, 55]. Briefly, U937/AST FPR stably transfected cells were labeled with red fluorescent PKH26GL dye, and B78H1/VCAM-1 transfectants were stained with green fluorescent PKH67GL dye (Sigma-Aldrich). Labeled cells were washed, resuspended in HEPES buffer supplemented with 0.1% HSA and stored on ice until used in assays. Control U937 cells were preincubated with the 1 μM LDV-containing small molecule for blocking adhesion. Prior to data acquisition, cells were warmed to 37° C. for 10 min separately and then mixed. During data acquisition, the samples were stirred with a 5×2-mm magnetic stir bar (Bel-Art Products, Pequannock, N.J.) at 300 rpm and kept at 37° C. For stimulation, cells were treated with appropriate GPCR ligands at saturating concentration (10 times or higher than K_{d}). In several experiments cells were treated sequentially with two different compounds. The number of cell aggregates containing U937 adherent to B78H1/VCAM-1 (red and green cofluorescent particles) as well as the number of singlets (red or green fluorescent particles, FL2 and FL1 in FACScan flow cytometer) were followed in real-time. The percentage of aggregates was calculated as follows: % U937 cells in aggregates=number of aggregates/(number of aggregates+number of U937 singlets))×100. Experiments were done using a FACScan flow cytometer and Cell Quest software (Becton Dickinson, San Jose, Calif.). The data were converted to number of singlets/aggregates versus time using FCSQuery software developed by Dr. Bruce Edwards (University of New Mexico).

### Statistical Analysis

Curve fits and statistics were performed using GraphPad Prism (GraphPad Prism version 4.00 for Windows, GraphPad Software, San Diego, Calif.). Each experiment was repeated at least three times. The experimental curves represent the mean of two or more independent runs. SEM was calculated using GraphPad Prism. To estimate the statistical significance of the difference between control and treated samples (as FIGS. 2B, 2C, 2D, 2E, and 3B, 3C), the sections of the kinetic curves at the peak of activation and after the steady state was reached (total of 30-80 seconds indicated on Figs. using “*” for the peak and “**” for the steady state) were compared using the unpaired t test (GraphPad Prism version 4.00 for Windows, GraphPad Software, San Diego, Calif.).

### Abbreviations

cAMP (adenosine 3′,5′-cyclophosphate), BAY 41-2272 (3-(4-Amino-5-cyclopropylpyrimidin-2-yl)-1-(2-fluorobenzyl)-1H-pyrazolo[3,4-b]pyridine, activator of soluble guanylate cyclase), DEA-NONOate (2-(N,N-Diethylamino)-diazenolate, nitric oxide donor), cGMP (guanosine 3′,5′-cyclic monophosphate), dbcGMP (N²,2′-O-Dibutyrylguanosine 3′,5′-cyclic monophosphate), fMLFF (N-formyl-L-methionyl-L-leucyl-L-phenylalanyl-L-phenylalanine, formyl peptide), FPR (formyl peptide receptor 1), GC (guanylate cyclase, guanylyl cyclase), GPCR (guanine nucleotide binding protein coupled receptor), HSA (human serum albumin), HEPES (4-(2-hydroxyethyl)-1-piperazineethanesulfonic acid), IL-8/CXCL8 (Interleukin-8), LDV containing small molecule (4-((N′-2-methylphenyl)ureido)-phenylacetyl-L-leucyl-L-aspartyl-L-valyl-L-prolyl-L-alanyl-L-alanyl-L-lysine), LDV-FITC containing small molecule (4-((N′-2-methylphenyl)ureido)-phenylacetyl-L-leucyl-L-aspartyl-L-valyl-L-prolyl-L-alanyl-L-alanyl-L-lysine-FITC), MCF (mean channel fluorescence, equivalent of mean fluorescence intensity), PKG (cGMP-dependent protein kinase), SDF-1 (stromal cell-derived factor-1, CXCL12), VCAM-1 (vascular cell adhesion molecule 1, CD106), VLA-4 (very late antigen 4, CD49d/CD29, α₄β₁ integrin).

### REFERENCES

1. Hynes R O: Integrins: bidirectional, allosteric signaling machines. Cell 2002, 110:673-687.
2. Lapidot T, Petit I: Current understanding of stem cell mobilization: the roles of chemokines, proteolytic enzymes, adhesion molecules, cytokines, and stromal cells. Exp Hematol 2002, 30:973-981.
3. Lapidot T, Dar A, Kollet O: How do stem cells find their way home? Blood 2005, 106:1901-1910.
4. Carman C V, Springer T A: Integrin avidity regulation: are changes in affinity and conformation underemphasized? Curr Opin Cell Biol 2003, 15:547-556.
5. Kim M, Carman C V, Yang W, Salas A, Springer T A: The primacy of affinity over clustering in regulation of adhesiveness of the integrin {alpha}L{beta}2. J Cell Biol 2004, 167:1241-1253.
6. Shimaoka M, Takagi J, Springer T A: Conformational regulation of integrin structure and function. Annu Rev Biophys Biomol Struct 2002, 31:485-516.
7. Desgrosellier J S, Cheresh D A: Integrins in cancer: biological implications and therapeutic opportunities. Nat Rev Cancer 2010, 10:9-22.
8. Wong C H, Heit B, Kubes P: Molecular regulators of leucocyte chemotaxis during inflammation. Cardiovasc Res 2010, 86:183-191.
9. Wang H, Lim D, Rudd C E: Immunopathologies linked to integrin signalling. Semin Immunopathol 2010, 32:173-182.
10. Cai W, Chen X: Anti-angiogenic cancer therapy based on integrin alphavbeta3 antagonism. Anticancer Agents Med Chem 2006, 6:407-428.
11. De Meyer S F, Vanhoorelbeke K, Ulrichts H, Staelens S, Feys H B, Salles I, Fontayne A, Deckmyn H: Development of monoclonal antibodies that inhibit platelet adhesion or aggregation as potential anti-thrombotic drugs. Cardiovasc Hematol Disord Drug Targets 2006, 6:191-207.
12. O'connor P: Natalizumab and the role of alpha 4-integrin antagonism in the treatment of multiple sclerosis. Expert Opin Biol Ther 2007, 7:123-136.
13. Hilden T J, Nurmi S M, Fagerholm S C, Gahmberg C G: Interfering with leukocyte integrin activation—a novel concept in the development of anti-inflammatory drugs. Ann Med 2006, 38:503-511.
14. Chigaev A, Blenc A M, Braaten J V, Kumaraswamy N, Kepley C L, Andrews R P, Oliver J M, Edwards B S, Prossnitz E R, Larson R S et al.: Real time analysis of the affinity regulation of alpha 4-integrin. The physiologically activated receptor is intermediate in affinity between resting and Mn(2+) or antibody activation. J Biol Chem 2001, 276:48670-48678.
15. Gazitt Y: Homing and mobilization of hematopoietic stem cells and hematopoietic cancer cells are mirror image processes, utilizing similar signaling pathways and occurring concurrently: circulating cancer cells constitute an ideal target for concurrent treatment with chemotherapy and antilineage-specific antibodies. Leukemia 2004, 18:1-10.
16. Alon R, Kassner P D, Can M W, Finger E B, Hemler M E, Springer T A: The integrin VLA-4 supports tethering and rolling in flow on VCAM-1. J Cell Biol 1995, 128:1243-1253.
17. Chen L L, Whiny A, Lobb R R, Adams S P, Pepinsky R B: Multiple activation states of integrin alpha4beta1 detected through their different affinities for a small molecule ligand. J Biol Chem 1999, 274:13167-13175.
18. Feigelson S W, Grabovsky V, Winter E, Chen L L, Pepinsky R B, Yednock T, Yablonski D, Lobb R, Alon R: The Src kinase p56(lck) up-regulates VLA-4 integrin affinity. Implications for rapid spontaneous and chemokine-triggered T cell adhesion to VCAM-1 and fibronectin. J Biol Chem 2001, 276:13891-13901.
19. Laudanna C, Kim J Y, Constantin G, Butcher E: Rapid leukocyte integrin activation by chemokines. Immunol Rev 2002, 186:37-46.
20. Arnaout M A, Goodman S L, Xiong J P: Structure and mechanics of integrin-based cell adhesion. Curr Opin Cell Biol 2007, 19:495-507.
21. Chigaev A, Waller A, Amit O, Sklar L A: Galphas-coupled receptor signaling actively down-regulates alpha4beta1-integrin affinity: a possible mechanism for cell de-adhesion. BMC Immunol 2008, 9:26.
22. Walter U, Gambaryan S: Roles of cGMP/cGMP-dependent protein kinase in platelet activation. Blood 2004, 104:2609.
23. Ni H, Freedman J: Platelets in hemostasis and thrombosis: role of integrins and their ligands. Transfus Apher Sci 2003, 28:257-264.
24. Roberts W, Riba R, Homer-Vanniasinkam S, Farndale R W, Naseem K M: Nitric oxide specifically inhibits integrin-mediated platelet adhesion and spreading on collagen. J Thromb Haemost 2008, 6:2175-2185.
25. Wood J S, Yan X, Mendelow M, Corbin J D, Francis S H, Lawrence D S: Precision substrate targeting of protein kinases. The cGMP- and cAMP-dependent protein kinases. J Biol Chem 1996, 271:174-179.
26. Aicher A, Heeschen C, Mildner-Rihm C, Urbich C, Ihling C, Technau-Ihling K, Zeiher A M, Dimmeler S: Essential role of endothelial nitric oxide synthase for mobilization of stem and progenitor cells. Nat Med 2003, 9:1370-1376.
27. Aicher A, Heeschen C, Dimmeler S: The role of NOS3 in stem cell mobilization. Trends Mol Med 2004, 10:421-425.
28. Bonig H, Wundes A, Chang K H, Lucas S, Papayannopoulou T: Increased numbers of circulating hematopoietic stem/progenitor cells are chronically maintained in patients treated with the CD49d blocking antibody natalizumab. Blood 2008, 111:3439-3441.
29. Zohren F, Toutzaris D, Klarner V, Hartung H P, Kieseier B, Haas R: The monoclonal anti-VLA-4 antibody natalizumab mobilizes CD34+ hematopoietic progenitor cells in humans. Blood 2008, 111:3893-3895.
30. Papayannopoulou T, Nakamoto B: Peripheralization of hemopoietic progenitors in primates treated with anti-VLA4 integrin. Proc Natl Acad Sci USA 1993, 90:9374-9378.
31. Bonig H, Watts K L, Chang K H, Kiem H P, Papayannopoulou T: Concurrent blockade of alpha4-integrin and CXCR4 in hematopoietic stem/progenitor cell mobilization. Stem Cells 2009, 27:836-837.
32. Ramirez P, Rettig M P, Uy G L, Deych E, Holt M S, Ritchey J K, DiPersio J F: BIO5192, a small molecule inhibitor of VLA-4, mobilizes hematopoietic stem and progenitor cells. Blood 2009, 114:1340-1343.
33. Mendelsohn M E: Viagra: now mending hearts. Nat Med 2005, 11:115-116.
34. Thom S R, Bhopale V M, Mancini D J, Milovanova T N: Actin S-nitrosylation inhibits neutrophil beta2 integrin function. J Biol Chem 2008, 283:10822-10834.
35. Walsh G M, Leane D, Moran N, Keyes T E, Förster R J, Kenny D, O'Neill S: S-Nitrosylation of platelet alphaIIbbeta3 as revealed by Raman spectroscopy. Biochemistry 2007, 46:6429-6436.
36. Friebe A, Koesling D: Regulation of nitric oxide-sensitive guanylyl cyclase. Circ Res 2003, 93:96-105.
37. Dangel O, Mergia E, Karlisch K, Groneberg D, Koesling D, Friebe A: Nitric oxide-sensitive guanylyl cyclase is the only nitric oxide receptor mediating platelet inhibition. J Thromb Haemost 2010, 8:1343-1352.
38. Keefer L K, Nims R W, Davies K M, Wink D A: “NONOates” (1-substituted diazen-1-ium-1,2-diolates) as nitric oxide donors: convenient nitric oxide dosage forms. Methods Enzymol 1996, 268:281-293.
39. Stasch J P, Becker E M, Alonso-Alija C, Apeler H, Dembowsky K, Feurer A, Gerzer R, Minuth T, Perzborn E, Pleiss U et al.: NO-independent regulatory site on soluble guanylate cyclase. Nature 2001, 410:212-215.
40. Straub A, Stasch J P, Alonso-Alija C, Benet-Buchholz J, Ducke B, Feurer A, Furstner C: NO-independent stimulators of soluble guanylate cyclase. Bioorg Med Chem Lett 2001, 11:781-784.
41. Schwede F, Maronde E, Genieser H, Jastorff B: Cyclic nucleotide analogs as biochemical tools and prospective drugs. Pharmacol Ther 2000, 87:199-226.
42. Chigaev A, Waller A, Zwartz G J, Buranda T, Sklar L A: Regulation of cell adhesion by affinity and conformational unbending of alpha4beta1 integrin. J Immunol 2007, 178:6828-6839.
43. Chigaev A, Waller A, Amit O, Halip L, Bologa C G, Sklar L A: Real-time Analysis of
Conformation-sensitive Antibody Binding Provides New Insights into Integrin Conformational Regulation. J Biol Chem 2009, 284:14337-14346.
44. Njus B H, Chigaev A, Waller A, Wlodek D, Ostopovici-Halip L, Ursu O, Wang W, Oprea T I, Bologa C G, Sklar L A: Conformational mAb as a tool for integrin ligand discovery. Assay Drug Dev Technol 2009, 7:507-515.
45. Lin K, Ateeq H S, Hsiung S H, Chong L T, Zimmerman C N, Castro A, Lee W C, Hammond C E, Kalkunte S, Chen L L et al.: Selective, tight-binding inhibitors of integrin alpha4beta1 that inhibit allergic airway responses. J Med Chem 1999, 42:920-934.
46. Chigaev A, Zwartz G, Graves S W, Dwyer D C, Tsuji H, Foutz T D, Edwards B S, Prossnitz E R, Larson R S, Sklar L A: Alpha4beta1 integrin affinity changes govern cell adhesion. J Biol Chem 2003, 278:38174-38182.
47. Chigaev A, Buranda T, Dwyer D C, Prossnitz E R, Sklar L A: FRET detection of cellular alpha4-integrin conformational activation. Biophys J 2003, 85:3951-3962.
48. Prossnitz E R: Desensitization of N-formylpeptide receptor-mediated activation is dependent upon receptor phosphorylation. J Biol Chem 1997, 272:15213-15219.
49. Martinez S E, Wu A Y, Glavas N A, Tang X B, Turley S, Hol W G, Beavo J A: The two GAF domains in phosphodiesterase 2A have distinct roles in dimerization and in cGMP binding. Proc Natl Acad Sci USA 2002, 99:13260-13265.
50. Rybalkin S D, Rybalkina I G, Shimizu-Albergine M, Tang X B, Beavo J A: PDE5 is converted to an activated state upon cGMP binding to the GAF A domain. EMBO J 2003, 22:469-478.
51. Mullershausen F, Friebe A, Feil R, Thompson W J, Hofmann F, Koesling D: Direct activation of PDE5 by cGMP: long-term effects within NO/cGMP signaling. J Cell Biol 2003, 160:719-727.
52. Murthy K S: Activation of phosphodiesterase 5 and inhibition of guanylate cyclase by cGMP-dependent protein kinase in smooth muscle. Biochem J 2001, 360:199-208.
53. Murthy K S: Modulation of soluble guanylate cyclase activity by phosphorylation. Neurochem Int 2004, 45:845-851.
54. Edwards B S, Kuckuck F W, Prossnitz E R, Okun A, Ransom J T, Sklar L A: Plug flow cytometry extends analytical capabilities in cell adhesion and receptor pharmacology. Cytometry 2001, 43:211-216.
55. Zwartz G, Chigaev A, Foutz T, Larson R S, Posner R, Sklar L A: Relationship between Molecular and Cellular Dissociation Rates for VLA-4/VCAM-1 Interaction in the Absence of Shear Stress. Biophys J 2004, 86:1243-1252.
56. Laudanna C, Alon R: Right on the spot. Chemokine triggering of integrin-mediated arrest of rolling leukocytes. Thromb Haemost 2006, 95:5-11.
57. Ley K, Laudanna C, Cybulsky M I, Nourshargh S: Getting to the site of inflammation: the leukocyte adhesion cascade updated. Nat Rev Immunol 2007, 7:678-689.
58. Li Z, Delaney M K, O'Brien K A, Du X: Signaling during platelet adhesion and activation. Arterioscler Thromb Vasc Biol 2010, 30:2341-2349.
59. Cox D, Brennan M, Moran N: Integrins as therapeutic targets: lessons and opportunities. Nat Rev Drug Discov 2010, 9:804-820.
60. Sklar L A, Hyslop P A, Oades Z G, Omann G M, Jesaitis A J, Painter R G, Cochrane C G: Signal transduction and ligand-receptor dynamics in the human neutrophil. Transient responses and occupancy-response relations at the formyl peptide receptor. J Biol Chem 1985, 260:11461-11467.
61. Sklar L A, Omann G M: Kinetics and amplification in neutrophil activation and adaptation. Semin Cell Biol 1990, 1:115-123.
62. Chigaev A, Wu Y, Williams D B, Smagley Y, Sklar L A: Discovery of very late antigen-4 (VLA-4, {alpha}4{beta}1 integrin) allosteric antagonists. J Biol Chem 2011, 286:5455-5463.
63. Ervens J, Seifert R: Differential modulation by N4,2′-O-dibutyryl cytidine 3′:5′-cyclic monophosphate of neutrophil activation. Biochem Biophys Res Commun 1991, 174:258-267.
64. Desch M, Schinner E, Kees F, Hofmann F, Seifert R, Schlossmann J: Cyclic cytidine 3′,5′-monophosphate (cCMP) signals via cGMP kinase I. FEBS Lett 2010, 584:3979-3984.
65. Gaion R M, Krishna G: Cytidylate cyclase: possible artifacts in the methodology. Science 1979, 203:672-673.
66. Gottle M, Dove S, Kees F, Schlossmann J, Geduhn J, Konig B, Shen Y, Tang W J, Kaever V, Seifert R: Cytidylyl and uridylyl cyclase activity of bacillus anthracis edema factor and Bordetella pertussis CyaA. Biochemistry 2010, 49:5494-5503.
67. Schwarz U R, Walter U, Eigenthaler M: Taming platelets with cyclic nucleotides. Biochem Pharmacol 2001, 62:1153-1161.
68. Dustin M L, Springer T A: T-cell receptor cross-linking transiently stimulates adhesiveness through LFA-1. Nature 1989, 341:619-624.
69. Chigaev A, Smagley Y, Zhang Y, Waller A, Haynes M K, Amit O, Wang W, Larson R S, Sklar L A: Real-time analysis of the inside-out regulation of lymphocyte function-associated antigen-1 revealed similarities and differences with very late antigen-4. J Biol Chem 2011. Apr. 22. [Epub ahead of print] PMID:21515675.
70. Spina D: PDE4 inhibitors: current status. Br J Pharmacol 2008, 155:308-315.
71. Diamant Z, Spina D: PDE4-inhibitors: A novel, targeted therapy for obstructive airways disease. Pulm Pharmacol Ther 2011. [Epub ahead of print] PMID:21255672
72. Schafer P H, Parton A, Gandhi A K, Capone L, Adams M, Wu L, Bartlett J B, Loveland M A, Gilhar A, Cheung Y F et al.: Apremilast, a cAMP phosphodiesterase-4 inhibitor, demonstrates anti-inflammatory activity in vitro and in a model of psoriasis. Br J Pharmacol 2010, 159:842-855.
73. Kew R R, Peng T, DiMartino S J, Madhavan D, Weinman S J, Cheng D, Prossnitz E R: Undifferentiated U937 cells transfected with chemoattractant receptors: a model system to investigate chemotactic mechanisms and receptor structure/function relationships. J Leukoc Biol 1997, 61:329-337.
74. Osborn L, Hession C, Tizard R, Vassallo C, Luhowskyj S, Chi-Rosso G, Lobb R: Direct expression cloning of vascular cell adhesion molecule 1, a cytokine-induced endothelial protein that binds to lymphocytes. Cell 1989, 59:1203-1211.

1. A method of treating a subject who suffers from a VLA-4-related cell adhesion disorder, the method comprising administering to the subject a pharmaceutically-effective amount of a nitric oxide/cGMP signaling pathway modulator selected from the group consisting of a nitric oxide donor, a nitric oxide-independent activator of soluble guanylyl cyclase, or a cell permeable analog of cGMP.

2. The method of claim 1, wherein:
(a) the nitric oxide (NO) donor is selected from the group consisting of (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazeniumdiolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF;
(b) the nitric oxide-independent activator of soluble guanylyl cyclase is selected from the group consisting of BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin; and
(c) the cell permeable analog of cGMP is selected from the group consisting of N2,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

3. The methods of claim 1, wherein the VLA-4-related cell adhesion disorder is selected from the group consisting of multiple sclerosis, meningitis, encephalitis, stroke, other cerebral traumas, inflammatory bowel disease including ulcerative colitis and Crohn's disease, rheumatoid arthritis, asthma, acute juvenile onset diabetes (Type 1), AIDS dementia, atherosclerosis, nephritis, retinitis, atopic dermatitis, psoriasis, myocardial ischemia, acute leukocyte-mediated lung injury such as occurs in adult respiratory distress syndrome, tumor metastasis, transplant rejection, graft versus host disease, melanoma, multiple myeloma, malignant lymphoma, acute and chronic leukemias, pancreatic cancer, neuroblastoma, small cell and non-small cell lung cancer, mesothelioma, colorectal carcinoma, and breast cancer.

4. The methods of claim 1, wherein the subject is co-administered a combination of at least two active ingredients selected from the group consisting of a nitric oxide donor, a nitric oxide-independent activator of soluble guanylyl cyclase, and a cell permeable analog of cGMP.

5. The method of claim 4, wherein the VLA-4-related cell adhesion disorder is selected from the group consisting of multiple sclerosis, meningitis, encephalitis, stroke, other cerebral traumas, inflammatory bowel disease including ulcerative colitis and Crohn's disease, rheumatoid arthritis, asthma, acute juvenile onset diabetes (Type 1), AIDS dementia, atherosclerosis, nephritis, retinitis, atopic dermatitis, psoriasis, myocardial ischemia, acute leukocyte-mediated lung injury such as occurs in adult respiratory distress syndrome, tumor metastasis, transplant rejection, graft versus host disease, melanoma, multiple myeloma, malignant lymphoma, acute and chronic leukemias, pancreatic cancer, neuroblastoma, small cell and non-small cell lung cancer, mesothelioma, colorectal carcinoma, and breast cancer.

6. The methods of claim 1, wherein:
(a) the VLA-4-related cell adhesion disorder is selected from the group consisting of tumor metastasis, melanoma, multiple myeloma, malignant lymphoma, acute and chronic leukemias, pancreatic cancer, neuroblastoma, small cell and non-small cell lung cancer, mesothelioma, colorectal carcinoma, and breast cancer; and
(b) the subject is co-administered an additional anti-cancer agent along with the nitric oxide/cGMP signaling pathway modulator.

7. The methods of claim 1, wherein:
(a) the VLA-4-related cell adhesion disorder is selected from the group consisting of tumor metastasis, melanoma, multiple myeloma, malignant lymphoma, acute and chronic leukemias, pancreatic cancer, neuroblastoma, small cell and non-small cell lung cancer, mesothelioma, colorectal carcinoma, and breast cancer;
(b) the subject is co-administered a combination of at least two active ingredients selected from the group consisting of a nitric oxide donor, a nitric oxide-independent activator of soluble guanylyl cyclase, and a cell permeable analog of cGMP; and
(c) the subject is also co-administered an additional anti-cancer agent along with the nitric oxide/cGMP signaling pathway modulator.

8. A method of treating a subject who has been diagnosed as suffering from at least one VLA-4-related cell adhesion disorder selected from the group consisting of multiple sclerosis, ulcerative colitis, Crohn's disease, rheumatoid arthritis, asthma, acute juvenile onset diabetes (Type 1), AIDS dementia, atopic dermatitis, psoriasis, nephritis, retinitis, acute leukocyte-mediated lung injury, transplant rejection, and graft versus host disease the method comprising treating the at least one VLA-4-related cell adhesion disorder by administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP.

9. The method of claim 8, wherein:
(a) the nitric oxide (NO) donor is selected from the group consisting of (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazeniumdiolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF;
(b) the nitric oxide-independent activator of soluble guanylyl cyclase is selected from the group consisting of BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin; and
(c) the cell permeable analog of cGMP is selected from the group consisting of N2,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

10. The method of claim 9, wherein the diagnosed VLA-4-related cell adhesion disorder is treated by administering to the subject one or more nitric oxide/cGMP signaling pathway modulators selected from the group consisting of BAY 41-2272, BAY 41-8543, BAY 58-2667 (cinaciguat), BAY 60-2770, BAY 63-2521, YC-1 (3-(5′-hydroxymethyl-2′-furyl)-1-benzylindazole), A-350619, A-344905, and A-778935.

11. A method of treating a subject who has been diagnosed as suffering from at least one VLA-4-related cell adhesion disorder selected from the group consisting of atherosclerosis and myocardial ischemia, the method comprising treating the at least one VLA-4-related cell adhesion disorder by administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP.

12. (canceled)

13. (canceled)

14. The method of claim 11, wherein the subject also suffers from an additional cardiac disorder selected from the group consisting of decompensated heart failure, arterial pulmonary hypertension, venous pulmonary hypertension, hypoxic pulmonary hypertension, thromboembolic pulmonary hypertension and miscellaneous pulmonary hypertension, and the additional cardiac disorder is treated by separately administering one of the nitric oxide/cGMP signaling pathway modulators.

15. A method of treating a subject who has been diagnosed as suffering from at least one VLA-4-related cell adhesion disorder selected from the group consisting of tumor metastasis, melanoma, multiple myeloma, malignant lymphoma, acute and chronic leukemias, pancreatic cancer, neuroblastoma, small cell and non-small cell lung cancer, mesothelioma, colorectal carcinoma, and breast cancer, the method comprising treating the at least one VLA-4-related cell adhesion disorder by administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP.

16. The method of claim 11, wherein:
(a) the nitric oxide (NO) donor is selected from the group consisting of (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazeniumdiolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF;
(b) the nitric oxide-independent activator of soluble guanylyl cyclase is selected from the group consisting of BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin; and
(c) the cell permeable analog of cGMP is selected from the group consisting of N2,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

17. The method of claim 11, wherein the diagnosed tumor metastasis, melanoma, multiple myeloma, malignant lymphoma, acute and chronic leukemias, pancreatic cancer, neuroblastoma, small cell and non-small cell lung cancer, mesothelioma, colorectal carcinoma, or breast cancer is treated by administering to the subject one or more nitric oxide/cGMP signaling pathway modulators selected from the group consisting of BAY 41-2272, BAY 41-8543, BAY 58-2667 (cinaciguat), BAY 60-2770, BAY 63-2521, YC-1 (3-(5′-hydroxymethyl-2′-furyl)-1-benzylindazole), A-350619, A-344905, and A-778935.

18. The method of claim 15, wherein an additional anti-cancer agent is co-administered to the subject.

19. A method of treating a subject who has been diagnosed as suffering from a non-metastatic cancer, the method comprising administering to the subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP to prevent metastasis of the cancer.

20. (canceled)

21. (canceled)

22. (canceled)

23. (canceled)

24. (canceled)

25. (canceled)

26. (canceled)

27. (canceled)

28. A method of determining whether a subject suffers from, or is at risk of developing VLA-4-related cell adhesion disorder, the method comprising determining a cyclic GMP (cGMP) level in a sample obtained from the subject and comparing the determined cyclic GMP (cGMP) level to a control cyclic GMP (cGMP) level, wherein a decrease in cyclic GMP (cGMP) level indicates an increased likelihood that the subject suffers from or is at risk of developing VLA-4-related cell adhesion disorder.

29. (canceled)

30. (canceled)

31. (canceled)

32. (canceled)

33. (canceled)

34. (canceled)

35. A pharmaceutical composition comprising:
(a) at least one nitric oxide/cGMP signaling pathway modulator as defined herein;
(b) at least one additional VLA-4 antagonist; and optionally
(c) a pharmaceutically-acceptable excipient.

36. (canceled)

37. The pharmaceutical composition according to claim 35 wherein:
(a) the nitric oxide (NO) donor is selected from the group consisting of (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazeniumdiolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF;
(b) the nitric oxide-independent activator of soluble guanylyl cyclase is selected from the group consisting of BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin; and
(c) the cell permeable analog of cGMP is selected from the group consisting of N2,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

38. The composition according to claim 35 wherein said VLA-4 antagonist is (natalizumab), AN-100226 (Antegren), CDP323, Firategrast, ATL/TV1102, ATL1102, clafrinast, RBx-7796, pharmaceutically acceptable salts and mixtures thereof.

39. A pharmaceutical composition comprising:
(a) at least one nitric oxide/cGMP signaling pathway modulator as defined herein;
(b) at least one additional anti-cancer agent; and optionally
(b) a pharmaceutically-acceptable excipient.

40. (canceled)

41. The pharmaceutical composition according to claim 39 wherein:
(a) the nitric oxide (NO) donor is selected from the group consisting of (1) a S-nitrosothiol selected from the group consisting of S-nitroso-glutathione (GSNO), S-nitroso-N-acetylpenicillamine (SNAP), LA810 and S-nitroso-N-valerylpenicillamine (SNVP) (2) a diazeniumdiolate (NONOate) selected from the group consisting of diethylamine NONOate (DEA/NO), SPER/NO, PROLI/NO, JS-K Glyceryl trinitrate (GTN, mitochondrial aldehyde dehydrogenase (mtADH), isosorbide mononitrate (ISMN), pentaerythrityl tetranitrate (PETN), sodium nitroprusside (SNP), and BiDil (isosorbide dinitrate with hydralazine, and (3) a NO donor hybrid drug selected from the group consisting of NCX4215, NCX4016, nipradiol (K-351), niro-prvastatin, SNO-diclofenac, SNO-captopril, furoxan bound to 4-phenyl-1,4-dihydropyridine, REC15/2739, SNO-t-PA and SNO-vWF;
(b) the nitric oxide-independent activator of soluble guanylyl cyclase is selected from the group consisting of BAY 41-2272, BAY 41-8543, BAY 58-2667 (cinaciguat), BAY 60-2770, BAY 63-2521, HMR-1766, YC-1 (3-(5′-hydroxymethyl-2′-furyl)-1-benzylindazole), CFM-1571, A-350619, A-344905, A-778935, 7-[2-[4-(2-methoxyphenyl)pipe-razinyl]-ethyl]-1,3-dimethylxanthine (KMUP-1); a porphyrin, and a metallopophyrin; and
(c) the cell permeable analog of cGMP is selected from the group consisting of N2,2′-O-dibutyrylguanosine 3′,5′-cyclic monophosphate, 8-bromo-cGMP, 8-chloroadenosine 3′,5′-cyclic monophosphate sodium salt, dibutyryl-cGMP, Rp-8-Br-cGMPS, 8-pCPT-cGMP, 2′-dcGMP, and 8-Br-PET-cGMP.

42. (canceled)

43. The composition according to claim 39 wherein said additional anti-cancer is agent is adriamycin, aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate, or a mixture thereof.

44. A method of regulating stem cell adhesion in a patent or subject in need, comprising administering to said patient or subject a pharmaceutically-effective amount of at least one nitric oxide/cGMP signaling pathway modulator selected from the group consisting of a nitric oxide donor, a nitric oxide-independent activator of soluble guanylyl cyclase, or a cell permeable analog of cGMP and optionally collecting, purifying, and/or transplanting said cells.

45. (canceled)

46. (canceled)
